(19) United States
(12) Patent Application Publication
(10) Pub. No.: US 2018/0021459 A1
(43) Pub. Date: Jan. 25, 2018

(54) DETERMINING THE CONDITION OF A WOUND

(71) Applicant: Microarray Limited, Bedford (GB)

(72) Inventors: Christopher David HUNT, Harpenden (GB); Paul James DAVIS, Sharnbrook (GB)

(21) Appl. No.: 15/550,352

(22) PCT Filed: Feb. 12, 2016
(86) PCT No.: PCT/GB2016/050342

§ 371 Date: Aug. 10, 2017

(30) Foreign Application Priority Data
Feb. 12, 2015 (GB) 1502350.0

(51) Int. Cl. A61K 49/00; C12Q 1/37; A61B 5/00; A61L 15/56; A61F 13/00; A61B 5/145

(52) U.S. Cl. CPC A61K 49/0004; A61F 13/00059; A61F 13/00068; A61B 5/14507; A61B 5/445; A61L 15/56; C12Q 1/37; C12Y 304/22002

(57) ABSTRACT

A product for monitoring the condition of the wound comprising a biologically inert matrix which absorbs wound exudate and one or more reagents on or in the matrix for measuring one or more markers comprised within the wound exudate. A change in the one or more reagents caused by the one or more markers comprised within the wound exudate provides a visual indication of an alteration in the condition of the wound. Companion wound dressings, kits and methods are also provided.

### FIELD OF THE INVENTION

The invention generally relates to products and associated methods for determining the condition of a wound, which may be a chronic wound.

### BACKGROUND TO THE INVENTION

A wound may be defined as a breakdown in the protective function of the skin; the loss of continuity of epithelium, with or without loss of underlying connective tissue (i.e. muscle, bone, nerves) following injury to the skin or underlying tissues/organs caused, for example, by surgery, a blow, a cut, chemicals, heat/cold, friction/shear force, pressure or as a result of disease, such as leg ulcers or carcinomas (Leaper and Harding, Wounds: Biology and Management, Oxford University Press (1998)).

Wound healing comprises restoration of any damaged tissue comprising formation of new connective tissues and re-growth of epithelium (Copper, A review of different wound types and their principles of management in Wound Healing: A systematic approach to advanced wound healing and management, Cromwell Press, UK (2005)).

Wounds can be classified as acute or chronic. Acute wounds comprise those in which healing occurs as a sequential cascade of overlapping processes that requires the coordinated completion of a variety of cellular activities. Conversely, a chronic wound is one in which the normal process of wound healing is disrupted at one or more points in the phases of wound healing. Often this may lead to a chronic wound becoming stuck in a particular phase of healing such as inflammation or proliferation. Chronic wounds are often identified by the presence of a raised, hyperproliferative, yet nonadvancing wound edge. The local wound environment, rich in inflammatory products, and proinflammatory cytokines may comprise an imbalanced enzymatic milieu consisting of an excess of matrix metalloproteases and a reduction in their inhibitors resulting in the destruction of the extracellular matrix (Menke et al., Impaired wound healing, Clinical Dermatology (2007)). The resultant profound inflammatory state is thought to be a significant factor influencing and delaying healing. Furthermore, chronic wounds can often become impeded by the accumulation of necrotic or sloughy tissue in the wound bed. It has been reported that, in the US alone, chronic wounds affect approximately 5.7 million patients and cost an estimated US$20 billion annually (Branski et al., A review of gene and stem cell therapy in cutaneous wound healing, Burns (2008)). Common chronic wounds include diabetic ulcers, vascular ulcers and pressure ulcers (Werdin et al., Evidence-based Management Strategies for Treatment of Chronic Wounds, Eplasty (2009)).

Management of wound healing has been suggested to comprise four principal elements: the tissue within and surrounding the wound and its status, the presence of any inflammation and/or infection within or surrounding the wound, the moisture balance within the wound and the quality of the wound edge (Ayello et al., TIME heals all wounds, Nursing (2004)).

At present, wound dressings are available which seek to address one or more of these principal elements. Choosing an appropriate wound dressing comprises consideration of the current phase of wound healing, its specific temporal requirements, as well as potential side effects. Ideally, dressings should minimize pain and be easy to use. These dressings must prevent friction and shear while protecting the peri-ulcer tissue and skin. A combination of different dressings at different stages of the healing process has been proposed. For instance, the use of hydrogel dressings for the debridement phase, foam dressings at the granulation stage, and the use of either hydrocolloids or low adherence dressings for the epithelialization phase (Vaneau et al., Consensus panel recommendations for chronic and acute wound dressings, Archives of Dermatology (2007)).

### SUMMARY OF THE INVENTION

Monitoring of the condition of the wound is currently limited to an initial assessment by the caregiver responsible for routine changing of the wound dressing comprising evaluation of one or more of the appearance and/or smell of the wound and/or the volume of exudate production. Such an assessment may suggest to the caregiver that referral of the patient to a clinical practitioner or further analysis of the wound and/or wound exudate is required.

However, such assessments are unable to analyse one or more components of the exudate at the point of care to indicate the condition of the wound. Furthermore, there currently exists no means by which to monitor the condition of the wound without removal of the wound dressing, with such removal being generally limited to routine visits by the caregiver at the point of care.

Accordingly, the invention provides a product for monitoring the condition of a wound comprising, consisting essentially of or consisting of:
(i) a biologically inert matrix which absorbs wound exudate; and
(ii) one or more reagents on or in the matrix for measuring one or more markers comprised within the wound exudate

wherein a change in the one or more reagents caused by the one or more markers comprised within the wound exudate provides a visual indication of an alteration in the condition of the wound.

“Wound” can be defined as a breakdown in the protective function of the skin; the loss of continuity of epithelium, with or without loss of underlying connective tissue (i.e. muscle, bone, nerves) following injury to the skin or underlying tissues/organs caused, for example, by surgery, a blow, a cut, chemicals, heat/cold, friction/shear force, pressure or as a result of disease, such as leg ulcers or carcinomas.

“Wound exudate” should be understood to mean the fluid environment of the wound which is exposed to the external environment by virtue of the breakdown in the protective function of the skin and loss of continuity of epithelium comprising pus, serum, water and/or blood and further comprising one or more lipids, polysaccharides, proteins, in particular proteases such as extracellular matrix proteins including collagenases (more specifically, for example, gelatinases), and cellular debris.

“Biologically inert matrix” should be understood to mean a matrix as further defined herein which does not interact with or initiate a response from biological tissue with which it comes into contact.

Central to the invention is the fact that the product provides a complete test unit and thus provides a direct indication of wound status without requiring any further downstream processing. Thus, in particular embodiments, the one or more reagents form a test unit on or in the matrix. The test unit is exposed to wound exudate as it is absorbed from the wound by the matrix and the visual indication is provided by the test unit as a consequence of a modification in the one or more reagents comprising the test unit caused by one or more markers comprised within the wound exudate. In further embodiments, the product may comprise more than one test unit on or in the matrix. The more than one test units may comprise the same or different one or more reagents. Consequently, the more than one test units may detect and measure same or different one or more markers present in the absorbed wound exudate. In further embodiments, the one or more reagents may form a discrete reaction zone on or within the matrix. The one or more reagents are exposed to wound exudate as it is absorbed from the wound and the visual indication is provided in the reaction zone portion of the matrix. In alternative embodiments, the one or more reagents are dispersed throughout the matrix.

In certain embodiments, the visual indication generated as a consequence of a modification of the one or more reagents, as further described herein, by a marker present in the wound exudate absorbed by the matrix indicates that the condition of the wound has deteriorated. The visual indication may indicate an improvement in healing of the wound in some embodiments. It is also possible to include both a deterioration and an improvement marker in the matrix in some embodiments. They may be contained in separate test units, or reaction zones, within the matrix to clearly delineate the visual indications from one another.

In further embodiments, the matrix is able to absorb and retain a volume of exudate sufficient for further (downstream and separate) analysis of the exudate as further described herein. For instance, in certain embodiments, the matrix has the capacity to absorb a volume of at least 0.2 ml (wound exudate). In further embodiments, the matrix has the capacity to absorb a volume in the range of 0.2 ml to 10 ml. For instance, a volume of at least 0.2 ml, 0.3 ml, 0.4 ml, 0.5 ml, 1 ml, 2 ml, 3 ml, 4 ml, 5 ml or 10 ml. In a particular embodiment, the matrix has the capacity to absorb a volume of 3 ml (to include a range of 2.5 to 3.4 ml).

Where the matrix performs dual functions (i.e. provides a visual indicator of an alteration in the condition of the wound and also retains a volume of exudate sufficient for further analysis) the matrix may be comprised of a first and second portion. Accordingly, throughout the disclosure, reference to “matrix” encompasses reference to first and/or second portions of a matrix. The matrix may thus comprise first and second layers.

These two portions or layers may be attached to each other, for instance, by lamination. Each portion has one or more or all of the features of the matrix as described herein. Each portion may comprise the same or different one or more markers described herein on or in that portion of the matrix.

In some embodiments, the first portion comprises the one or more reagents described herein, which may form a test unit as described herein, on or in that portion of the matrix. The first portion is able to absorb sufficient wound exudate to allow the one or more reagents comprised on or within its substance to come into contact with the wound exudate and, therefore, the one or more markers that may be contained therein.

In some embodiments, the second portion is able to absorb wound exudate in an amount sufficient for downstream analysis of the wound exudate as described further herein.

Accordingly, the invention provides a product for monitoring the condition of a wound comprising, consisting essentially of or consisting of:
(i) a first (biologically inert) matrix which absorbs wound exudate and comprises one or more reagents on or in the matrix for measuring one or more markers comprised within the wound exudate, wherein a change in the one or more reagents caused by the one or more markers comprised within the wound exudate provides a visual indication of an alteration in the condition of the wound; and
(ii) a second (biologically inert) matrix which absorbs wound exudate in an amount sufficient for downstream analysis of the wound exudate.

The matrix may thus be arranged such that the second portion comes directly into contact with the wound and the first portion indirectly absorbs wound exudate through fluid communication with the second portion. The first portion may thus be stacked on top of the second portion. The pressure applied by a wound dressing may keep the portions in fluid connect in situ. In other embodiments they may be more permanently connected, such as by lamination.

In further embodiments, the surface of the first portion that is not in contact with the second portion is coated with or otherwise surrounded by a transparent film in order to protect it from physical damage. Wound exudate absorbed by the second portion may still access the first portion via connecting surface that permits fluid communication. Such an arrangement ensures that the one or more reagents comprised on or within the first portion of the matrix are exposed to the wound exudate. Transparency of the film allows any signal generated as a consequence of the one or more reagents coming into contact with the one or more markers in the exudate, as further described herein, to be detected visually at the point of care.

In some embodiments, the first portion or layer of the matrix is substantially thinner than the second portion. In some embodiments, the first portion or layer comprises a membrane. In some embodiments, the second portion comprises an absorbent foam material, such as polyurethane foam.

It will be appreciated that the specific dimensions of a wound are unique in each case and that wound dressing size is adapted accordingly, for example using cut to size dressings. Consequently, in a further embodiment, the matrix has, or portions of the matrix have, dimensions suitable for and intended to facilitate positioning of the product between a wound dressing and the wound. In certain embodiments, the matrix, or first and/or second portions of the matrix, has thickness×width×length dimensions of at least 2 mm×10 mm×10 mm but not more than 7 mm×40 mm×40 mm. In a particular embodiment, the matrix has the dimensions 5 mm×25 mm×25 mm. The top and bottom surfaces do not necessarily have to be square in all embodiments. They could be rectangular for example. The matrix, or portions thereof, may be cylindrical in some embodiments. The matrix may also be provided in a cut to size format, provided each matrix once cut provided the one or more reagents on or in the matrix (e.g. in a test unit or reaction zone). The matrix is sufficiently soft and comfortable to minimise or avoid causing significant discomfort in the wound, particularly after the wound dressing has been applied. Application of the wound dressing exerts a level of compression on the matrix. Accordingly, in further embodiments, the matrix is sufficiently resistant to compression to allow the matrix, or portion thereof, to maintain a structure suitable to absorb sufficient volumes of wound exudate for further testing. Suitable volumes to be absorbed are discussed hereinabove. Thus, the matrix will inevitably be compressed to an extent underneath the wound dressing. However, this should still allow the matrix to absorb sufficient quantities of wound exudate for the downstream testing.

The matrix is biologically inert. Consequently, the matrix does not measurably alter the condition of the wound exudate or its components once absorbed.

The product may further comprise a reaction vessel extending from the matrix and which is in fluid connection with the (remainder of the) matrix. In certain embodiments, the reaction vessel is connected with the matrix via one or more capillary flow paths. As a consequence, the reaction vessel is exposed to wound exudate absorbed by the matrix once the product is in contact with a wound. Thus, in certain embodiments, the reaction vessel absorbs wound exudate indirectly via the matrix.

In a preferred embodiment, the reaction vessel extends sufficiently from the matrix to be positioned outside of a wound dressing applied to the wound such that it is visible to the subject suffering from the wound or to the caregiver. A caregiver is any person responsible for changing the wound dressing and inspecting the wound, for instance a district nurse or family member.

In certain embodiments, the reaction vessel incorporates one or more reagents, as further described herein, for measuring one or more markers comprised within the wound exudate wherein a change in the one or more reagents caused by one or more markers comprised within the wound exudate provides a visual indication of an alteration in the condition of the wound. The one or more reagents incorporated in the reaction vessel may be the same or different to those on or in the matrix. In some embodiments, the one or more reagents are in replacement of the matrix reagents. Thus, it is envisaged that the reaction vessel may provide the only visual indication of an alteration in the condition of the wound. In such embodiments, the remainder of the matrix to which the reaction vessel is connected, provides the role of exudate absorbent only. This has the benefit that the matrix that absorbs the exudate for downstream testing does not contain any additional reagents.

Thus, in embodiments in which the one or more reagents incorporated in the reaction vessel are different to those on or in the matrix, different markers comprised within the wound exudate may be detected and measured by each reagent set respectively. In other embodiments, the same marker is detected but using a different reagent system.

This may be the case for example where a biocompatible reagent is included in the matrix but a non-biocompatible reagent is included in the separate reaction vessel (because the reaction vessel is not in direct contact with the wound), as discussed below.

In embodiments in which the reaction vessel extends sufficiently from the matrix to be positioned outside of a wound dressing applied to the wound such that it is visible to the subject suffering from the wound or to the caregiver, the subject suffering from the wound or the caregiver at the point of care can observe the visual indication signalling an alteration in the condition of the wound without needing to remove the wound dressing. As a result, the subject and/or caregiver is potentially able to receive earlier warning of a change in the condition of the wound, such as deterioration, and can therefore seek clinical input and/or intervention more quickly.

In particular embodiments, the reaction vessel is closed to the environment. An enclosed reaction vessel is advantageous to prevent wound exudate exposure outside of the wound dressing. This facilitates handling of the product also. In further embodiments, the reaction vessel comprises, consists essentially of or consists of an absorbent material contained within an impermeable housing. Optionally, the housing comprises a transparent window or the housing is transparent in order to allow the subject and/or caregiver at the point of care to observe the visual indication produced by the product following modification of the one or more reagents contained within the reaction vessel by one or more markers of the absorbed wound exudate.

In certain embodiments, the one or more reagents incorporated within the reaction vessel are not biocompatible and are contained within the reaction vessel in a manner so as not to be released into the matrix following exposure to wound exudate. This prevents dissociation of any bio-incompatible degradation products (as a consequence of interaction with one or more markers present in the wound exudate) into the wound site. For instance, in certain embodiments, the one or more reagents are covalently linked to the reaction vessel and remain so after interaction with one or more markers present in the wound exudate. For instance, additive oxidation of one or more covalently bound reagents by one or more markers present in the wound exudate. In further embodiments, the reaction vessel further comprises a one-way valve at the point of fluid connection with the matrix such that fluid that has entered the reaction vessel and components therein cannot escape back into the matrix.

The matrix is composed of a material suitable for application to a wound and for absorbing wound exudate (while under compression). In particular embodiments, the matrix is composed of a porous material. The matrix is typically provided as a sterile product.

In certain embodiments, the matrix is composed of one or more materials selected from:
(i) polyurethane; and/or
(ii) polyethylene; and/or
(iii) cellulose fibres; and/or
(iv) porous hydrophilic plastic.

Suitable porous hydrophilic plastics include those marketed by Porex Limited.

In particular embodiments where the matrix is comprised of a first and second portion as described herein, the first and second portion may be composed of the same or different materials. In some embodiments, the second portion is composed of polyurethane, which may be in the form of a foam. In particular embodiments, the polyurethane is a non-isocyanate based polyurethane.

The reagents included in the matrix (and/or reaction vessel in some embodiments) are processed or otherwise modified by one or more markers found within the wound exudate. In certain embodiments, the one or more reagents are insoluble in aqueous conditions.

In further embodiments, the one or more reagents comprise, consist essentially of or consist of a cross-linked polymer.

In certain embodiments, the one or more reagents may be dried into the matrix and/or conjugated to the matrix. In some embodiments, the one or more reagents are dried so as to form a defined test unit on or in the matrix. This may, for instance, be achieved by dispensing a solution containing the one or more reagents as a single droplet onto the matrix.

In particular embodiments, the change in the one or more reagents is degradation of the one or more reagents by the one or more markers, as further described herein, present in the wound exudate that has been absorbed by the product.

In a further embodiment, degradation of the one or more reagents reveals a visible symbol on or in the matrix which is otherwise visually concealed by the one or more reagents. In a particular embodiment, the one or more reagents comprise, consist essentially of or consist of collagen, optionally forming a collagen plaque, and the one or more markers comprises a collagenase. In such embodiments, degradation of the collagen by collagenase present in the wound exudate reveals an otherwise visually concealed symbol on or in the matrix. In further embodiments, the visible symbol is a printed visible symbol, for instance a cross.

In specific embodiments, the collagen is fully, substantially or partially denatured prior to use in the invention. Where this has occurred by partial hydrolysis of the collagen, it is termed “gelatin” as would be well-known to the person skilled in the art. In these specific embodiments where the one or more reagents comprise, consist essentially of or consist of gelatin, the one or more markers comprises a gelatinase. The skilled person will appreciate that a gelatinase may also be considered a collagenase. Equally, the skilled person will appreciate that collagenases are known in the art that are also able to function as a gelatinase. The skilled person is aware of, or readily able to determine using routine experimentation, suitable collagenases/gelatinases as appropriate.

In certain embodiments, the one or more reagents comprises or is:
(i) a protease substrate;
(ii) a myeloperoxidase substrate.

For instance, the one or more reagents may comprise one or more protease substrates, one or more myeloperoxidase substrates or a combination of at least one protease substrate and at least one myeloperoxidase substrate.

In particular embodiments, the one or more reagents is a substrate for matrix metalloprotease collagenase, human neutrophil elastase and/or papain-family enzymes, such as staphopain from Staphylococcus aureus, and comprises, consists essentially of or consists of collagen. In specific embodiments, the one or more reagents is a substrate for matrix metalloprotease gelatinase (such as MMP2, MMP8 or MMP9), human neutrophil elastase and/or papain-family enzymes, such as staphopain from Staphylococcus aureus, and comprises, consists essentially of or consists of gelatin. By “papain-family enzyme” is meant a member of the papain family of peptidases.

In further embodiments, the one or more reagents comprise, consist essentially of or consist of a substrate for a serine protease such as neutrophil elastase and comprise, consist essentially of or consist of elastin.

In further embodiments, the one or more reagents comprise, consist essentially of or consist of a substrate for a cathepsin protease such as cathepsin G.

In further embodiments, the one or more reagents comprise, consist essentially of or consist of labelled collagen. In specific embodiments, the one or more reagents comprise, consist essentially of or consist of labelled gelatin.

In certain embodiments, the myeloperoxidase substrate comprises a coloured dye that is oxidised by myeloperoxidase present in the wound exudate, such that the coloured dye molecules become bleached. In alternative embodiments, the coloured dye is a leuco-dye which would become coloured on oxidation by the action of the myeloperoxidase present in the wound exudate.

In particular embodiments, the label may comprise, consist essentially of or consist of a coloured collagen or gelatin substrate. In specific embodiments, the collagen may be labelled with activated carbon. This may be achieved by drying the collagen with activated carbon particles entrained within the dried collagen mass giving the labelled collagen a black colouration. Following cleavage and degradation of the labelled collagen by collagenase enzymes present in the wound exudate absorbed by the product, the black colouration reduces in intensity or disappears altogether. Similarly, in other embodiments, the collagen or gelatin may be dried with coloured micro-particles (which may be, for example, copper phthalocyanine tetrasulfonic acid tetrasodium salt or which are formed of, for instance, latex and/or polystyrene or any combination thereof) entrapped within the collagen or gelatin molecules giving the labelled collagen or gelatin the colouration of the micro-particles. Upon contact with the fluidic wound exudate the collagen or gelatin mass swells slightly and softens without dispersing and without the collagen or gelatin molecules being diluted into the bulk solution. Thus, the micro-particles remain entrapped within the collagen or gelatin until and unless the collagen or gelatin is cleaved by collagenase or gelatinase enzymes present in the wound exudate. Following cleavage and degradation of the labelled collagen or gelatin by collagenase or gelatinase enzymes present in the wound exudate absorbed by the product, the micro-particles can disperse and the colouration reduces in intensity or disappears altogether. The reduction in intensity is visually perceptible. In some embodiments, the label may comprise, consist essentially of or consist of one or more dye molecules chemically conjugated to the collagen so as to form coloured collagen molecules. Following cleavage and degradation of the dyed collagen by collagenase enzymes present in the wound exudate absorbed by the product, in particular when said enzymes are at or above a threshold level, the colour reduces in intensity or disappears altogether. The reduction in intensity is visually perceptible. In related embodiments, the product may first be prepared by dispensing a solution comprising, consisting essentially of or consisting of the coloured collagen molecules or coloured gelatin molecules as a singular droplet onto the matrix which is then dried to form a test unit on or in the matrix. These embodiments are not limited to collagen or gelatin but instead may apply to any relevant protease substrate which has appropriate protease sensitivity, drying characteristics and re-hydration properties. In some embodiments, a colour guide may be provided with the product to provide a reference for the expected colour changes/levels if the appropriate one or more markers, such as collagenase or gelatinase enzymes, are active in the exudate. This may be a scale, for example a simple scale of no, low or high activity, or a numerical scale in some embodiments.

In some embodiments, degradation of the coloured one or more reagents on or in the matrix as described herein reveals a visible symbol on or in the matrix which is otherwise visually concealed by the intact coloured one or more reagents as also described herein. Thus, in these embodiments, the product advantageously provides a dual indication of the presence of one or more markers in the wound exudate.

In further embodiments, the label may comprise, consist essentially of or consist of a fluorescent label that is quenched unless and until the collagen is cleaved by collagenase enzymes present in the wound exudate absorbed by the product. A suitable example is DQ™ Collagen (Catalog number: D12052, Life Technologies).

In certain embodiments, the one or more markers comprised within the exudate are enzymes capable of modifying the one or more reagents. For instance, in particular embodiments, the one or more enzymes are selected from:
(i) proteases; and/or
(ii) myeloperoxidase.

The one or more proteases may, in certain embodiments, be selected from one or more matrix metalloproteinases such as MMP2, MMP8 and/or MMP9. In particular embodiments, the one or more proteases may be collagenase, gelatinase and/or elastase enzymes. In further embodiments, the one or more enzymes is a serine protease such as neutrophil elastase, more particularly human neutrophil elastase. In yet further embodiments, the one or more enzymes is a cathepsin protease such as cathepsin G. In yet further embodiments, the one or more enzymes is a papain-family enzyme, such as staphopain from Staphylococcus aureus.

In one aspect of the invention, generation of the visual indication signalling that there has been an alteration in the condition of the wound via modification of the one or more reagents by one or more markers of the absorbed wound exudate indicates to the subject and/or caregiver, at the point of care, the need for further analysis of the exudate, as further described herein.

As a consequence, in certain embodiments, the absorbed exudate can be retrieved from the matrix for further analysis, for instance, by centrifugation of the matrix containing the absorbed wound exudate.

In certain embodiments, the change in the one or more reagents only occurs if the one or more markers are present at or above a pre-determined threshold level. Thus, the visual indication of the alteration in wound condition is triggered if the marker activity and/or concentration is altered from what would be expected in the healing wound. For example some collagenase activity would be expected in the wound, but an excess of activity (as discussed further herein) can indicate that the wound condition has deteriorated. For example, the pre-determined threshold level may be in the range 0.0001-0.1 mg/mL. For instance, it may be 0.0001 mg/mL, 0.001 mg/mL, 0.01 mg/mL, 0.0125 mg/mL, 0.025 mg/mL, 0.05 mg/mL or 0.1 mg/mL. In particular embodiments wherein the one or more markers comprises, consists essentially of or consists of a collagenase/gelatinase, a matrix metalloproteinase such as MMP2, MMP8 and/or MMP9, neutrophil elastase (optionally human neutrophil elastase) and/or papain-family enzymes, such as staphopain from Staphylococcus aureus, the pre-determined threshold level is 0.0001 mg/mL, 0.001 mg/mL, 0.01 mg/mL, 0.0125 mg/mL, 0.025 mg/mL, 0.05 mg/mL or 0.1 mg/mL.

While it is envisaged that the most advantageous application of the product of the invention is as a discrete product packaged entirely separately from a wound dressing, it is also possible to integrate the products of the invention into a wound dressing. Thus, the invention also provides a wound dressing incorporating a product of the invention as defined herein. The wound dressing and product may be provided in a kit of parts. Thus, the wound dressing incorporates the product of the invention when placing the wound dressing on the wound, as described in further detail herein.

It will be apparent to the skilled person that the product described herein can be designed or employed so as to absorb enough wound exudate to be able to provide a visual indication of a change in the wound without necessarily absorbing, or being able to absorb, sufficient wound exudate for further downstream processing as described herein. Thus, in certain embodiments, the product functions solely as an in-wound protease activity detector. These embodiments are advantageous as they are extremely simple to operate and interpret.

However, it will also be apparent to the skilled person from the present disclosure that a key aspect of many embodiments of the product of the invention is the ability to absorb sufficient wound exudate to enable further laboratory based testing of the exudate, in a remote setting from the subject. Once removed from the wound, the product then needs to be safely delivered to the laboratory in a manner such that the exudate remains diagnostically useful. Thus, the invention also provides a kit comprising a product as described herein and a vessel (suitable) for safe containment and shipping of the product. Following contact of the product with the wound and absorbance of wound exudate, the product can be removed from the wound and placed in the vessel to allow safe transportation to a laboratory for further analysis of the wound exudate, as further described herein.

In embodiments of the product wherein the matrix comprises a first and second portion as described herein, the first and second portion may be provided as two separate components in the kits described herein. In particular embodiments, these two components may be connectable to each other such that the user at the point of care can assemble the two components into a single unit for placing into contact with the wound (exudate). The wound dressing may retain the single unit in place. In certain embodiments, where the second portion is able to and has absorbed wound exudate in an amount sufficient for downstream analysis of the wound exudate as described further herein, only this second portion need be safely delivered to the laboratory as described herein. Thus, the second portion may be detachable from the first. Alternatively, the whole unit may be sent for further testing. The test result in the first portion provides useful diagnostic information so it may be advantageous to also transmit this.

In certain embodiments, at least the internal surface(s), and generally all surfaces to include external surfaces, of the containment vessel are biologically inert.

In further embodiments, contact of the matrix with the internal surface or surfaces of the containment vessel does not measurably alter the condition of the exudate or its components (markers).

The vessel should be sealable to enable safe transport without risk of the exudate leaking. The seal may be reversible or may need to be broken at the laboratory in order to gain access to the exudate. The vessel is typically a consumable single use item. It may be made of plastic in some embodiments. It may, however, be reusable following suitable sterilisation e.g. by autoclaving in some embodiments.

In another aspect, the invention provides a method for monitoring the condition of a wound on a subject comprising, consisting essentially of or consisting of:
(a) placing a product of the invention as described herein in contact with the wound under a wound dressing;
(b) leaving the product in contact with the wound for a pre-determined amount of time;
(c) determining the presence or absence of a visual indication of an alteration in the condition of the wound by the product;

wherein the presence of the visual indication signals the need for further analysis of the wound exudate.

In embodiments of the product wherein the matrix comprises a first and second portion as described herein, the first and second portions may be separate components, not connected to each other and independently placed in contact with the wound for a pre-determined amount of time. In other embodiments, the first and second portions may be provided as separate components which are connectable with one another and assembled by the user at the point of care into a single unit. This single unit is then placed in contact with the wound for a pre-determined amount of time. Thus, the second portion may be detachable from the first. Alternatively, the whole unit may be sent for further testing. The test result in the first portion provides useful diagnostic information so it may be advantageous to also transmit this. In particular embodiments, the first portion comprises the one or more reagents described herein, which may form a test unit as described herein, on or in that portion of the matrix. The first portion is able to absorb sufficient wound exudate to expose the one or more reagents comprised on or within the first portion to the wound exudate and, therefore, the one or more markers that may be contained therein. Thus, this first portion provides the visual indication of an alteration in the condition of the wound by the product as described herein. The second portion is able to absorb wound exudate in an amount sufficient for downstream analysis of the wound exudate as described further herein.

In certain embodiments, however, the visual indication is combined with one or more indications selected from:
(i) the smell of the wound
(ii) the total volume of exudate
(iii) the appearance of the wound
(iv) the systemic condition of the subject in order to determine the need for further analysis of the wound exudate.

The absence of a visual indication by the product may be offset by the presence of one or more of the other indications listed above which, when assessed collectively, determines that further analysis of the wound exudate is needed. Such assessment may be made by a caregiver, such as a district nurse, at the point of care or a clinician.

In a further embodiment, the method further comprises:
(d) removal of the product from contact with the wound;
(e) retrieving the exudate absorbed by the product;
(f) analysing the retrieved exudate in order to determine the condition of the wound.

In certain embodiments, wherein the product employed in the method comprises a reaction vessel as described herein, the reaction vessel is used to remove the product from the wound. This prevents direct contact with the matrix itself and, thus, reduces the risk of contamination of the absorbed wound exudate. In particular embodiments, the reaction vessel can be cleaved from the matrix, for instance at or near to the junction at which the reaction vessel extends from the matrix, thereby further minimising the possibility of contamination of the wound exudate absorbed by the matrix. Alternatively, removal of the product may utilise forceps or another instrument to prevent direct human contact with the matrix.

In specific embodiments, wherein the product comprises a matrix comprising a first and second portion as described herein, the wound exudate is retrieved from the second matrix portion which has absorbed an amount of wound exudate sufficient for downstream analysis of the wound exudate as described further herein.

In certain embodiments, step (d) further comprises storage and shipping of the product to a laboratory before step (e) is performed. For embodiments of the product wherein the matrix comprises a first and second portion as described herein, in certain embodiments only the second matrix portion is stored and shipped to the laboratory as it is this portion which has absorbed an amount of wound exudate sufficient for downstream analysis of the wound exudate as described further herein. In other embodiments, both the first and second matrix portion are stored and shipped to the laboratory so that, for instance, the extent of degradation of the one or more markers present in the first matrix portion can be evaluated in the laboratory. Thus, the second portion may be detachable from the first. Alternatively, the whole unit may be sent for further testing. The test result in the first portion provides useful diagnostic information so it may be advantageous to also transmit this. Storage of the product may be in a vessel as described herein suitable for safe containment and shipping prior to steps (e) and (f). In certain embodiments, the internal and external surfaces of the containment vessel are biologically inert. In further embodiments, contact of the matrix with the internal surface of the containment vessel does not measurably alter the condition of the exudate or its components.

In a further embodiment, the vessel containing the product removed from the wound is transported to a laboratory for further analysis of the absorbed wound exudate.

Retrieval of the exudate may be by any suitable means. The matrix may be squeezed to release the exudate or may be centrifuged for example.

Analysis of the retrieved exudate comprises one or more tests to characterise the condition of the wound. Such tests may facilitate treatment and selection of therapeutic and other clinical interventions. Any suitable test may be employed as would be readily understood by one skilled in the art. In certain embodiments, analysis of the retrieved exudate comprises, consists essentially of or consists of measuring the levels and/or activities of one or more of:
(i) N-terminal serum type 11 procollagen (P1NP)
(ii) N-acetyl-Proline-Glycine-Proline (acPGP)
(iii) a biomarker of neutrophil infiltration

N-terminal serum type 1 procollagen (P1NP) is an indicator molecule of collagen synthesis. N-acetyl-Proline-Glycine-Proline (acPGP) is a degradation product of collagen produced as a consequence of proteolytic cleavage of collagen by matrix metalloproteinases, in particular collagenase. Consequently, in a further embodiment, the levels of P1NP and acPGP are used to determine a Healing Index Ratio wherein:
(i) equal levels of P1NP and acPGP indicate healing and/or successful treatment of the wound because the rates of collagen synthesis and degradation are approximately in balance;
(ii) higher levels of acPGP relative to P1NP indicates inflammation and/or increased risk of infection within the wound because more collagen is being degraded than is being synthesised;
(iii) moderately higher levels of P1NP relative to acPGP indicates a strong healing trajectory because more collagen is being synthesised than is being degraded;
(iv) significantly higher levels of P1NP relative to acPGP indicates an increased risk of hypergranulation in the wound because excessive amounts of collagen are being synthesised relative to the rates of collagen degradation.

“Hypergranulation” is to be understood as the excessive deposition of granulation tissue that may extend above the wound margin and comprises newly formed collagen, elastin and capillary networks.

During wound healing, infiltrating neutrophils are recruited to the wound site and are involved in tissue degradation and tissue formation. As such, an excessive or reduced influx or activation of infiltrating neutrophils into the damaged tissue may have profound effects on downstream cell migration, proliferation, differentiation, and ultimately the quality of the healing response. In particular, calprotectin is a protein produced by neutrophils known to be present in plasma and markedly elevated in inflammatory conditions. Thus, in certain embodiments, the biomarker of neutrophil infiltration is calprotectin.

In further embodiments, analysis of the retrieved exudate further or alternatively comprises, consists essentially of or consists of measuring the levels of one or more of:
(i) Hydroxylation of lysine and proline residues free in the wound exudate;
(ii) angiogenesis biomarkers; and/or
(iii) blood vessel differentiation biomarkers

In certain embodiments, low levels of hydroxylation of lysine and proline residues free in the wound exudate indicate the need for treatment to promote increased blood flow and access of oxygen to the wound.

In certain embodiments, the angiogenesis biomarkers comprise, consist essentially of or consist of vascular endothelial growth factor.

In certain embodiments, the blood vessel differentiation biomarkers comprise, consist essentially of or consist of intercellular adhesion molecule.

In certain embodiments, low levels of vascular endothelial growth factor and/or intercellular adhesion molecule indicate to treat the wound with externally applied vascular endothelial growth factor supplements.

In further embodiments, analysis of the retrieved exudate further or alternatively comprises, consists essentially of or consists of determining the nitric oxide (NO) status of the wound by measuring the levels and/or activities of one or more of:
(i) inducible nitric oxide synthase
(ii) carboxymethyllysine
(iii) arginase

As understood in the art, a lack of inducible nitric oxide synthase would disable NO responses normally needed in physiological homeostasis. Similarly, accumulation of carboxymethyllysine indicates that the wound site is in a NO deficient state. It will also be appreciated that arginase consumes arginine in a metabolic pathway which does not produce NO and is a competitor to NO synthase.

A low nitric oxide status indicates to treat the subject suffering from the wound with nitric oxide therapy or an arginine dietary supplement.

In further embodiments, analysis of the retrieved exudate further comprises, consists essentially of or consists of measuring the levels of one or more of:
(i) desmosine
(ii) matrix metalloproteinase (MMP8)
(iii) calprotectin
(iv) TIMP1
(v) TIMP2
(vi) A1AT
(vii) interleukin 6

By marker levels is meant the level of expression and/or activity and/or amount and/or concentration of the marker in the wound exudate. Expression levels may correlate with activity and can thus be used as a surrogate of activity and vice versa.

The person skilled in the art will be familiar with techniques and methods to determine the level of P1NP , acPGP, hydroxylation of lysine and proline residues, angiogenesis, vascular endothelial growth factor, blood vessel differentiation, intercellular adhesion molecule, inducible nitric oxide synthase, carboxymethyllysine, arginase, desmosine, MMP8, calprotectin, TIMP1, TIMP2, A1AT or interleukin 6.

For instance, expression levels may be measured at the level of protein or mRNA according to any suitable method. Protein modifications, such as glycosylation may also be relevant and can be measured by any suitable method. Many such methods are well known in the art and include use of mass spectrometry (e.g. MALDI-TOF mass spectrometry).

The expression level and/or amount and/or concentration of a marker (e.g. a protein) may rely upon a binding reagent such as an antibody or aptamer that binds specifically to the marker of interest (e.g. protein). The antibody may be of monoclonal or polyclonal origin. Fragments and derivative antibodies may also be utilised, to include without limitation Fab fragments, ScFv, single domain antibodies, nanoantibodies, heavy chain antibodies, aptamers etc. which retain specific binding function and these are included in the definition of “antibody”. Such antibodies are useful in the methods of the invention. They may be used to measure the level of a particular marker (e.g. protein, or in some instances one or more specific isoforms of a protein. The skilled person is well able to identify epitopes that permit specific isoforms to be discriminated from one another).

Methods for generating specific antibodies are known to those skilled in the art. Antibodies may be of human or non-human origin (e.g. rodent, such as rat or mouse) and be humanized etc. according to known techniques (Jones et al., Nature (1986) May 29Jun. 4; 321(6069):522-5; Roguska et al., Protein Engineering, 1996, 9(10):895-904; and Studnicka et al., Humanizing Mouse Antibody Frameworks While Preserving 3-D Structure. Protein Engineering, 1994, Vol.7, pg 805).

In certain embodiments the expression level and/or amount and/or concentration of a marker is determined using an antibody or aptamer conjugated to a label. By label is meant a component that permits detection, directly or indirectly. For example, the label may be an enzyme, optionally a peroxidase, or a fluorophore.

A label is an example of a detection agent. By detection agent is meant an agent that may be used to assist in the detection of the antibody-marker (e.g. protein) complex. Where the antibody is conjugated to an enzyme the detection agent may comprise a chemical composition such that the enzyme catalyses a chemical reaction to produce a detectable product. The products of reactions catalysed by appropriate enzymes can be, without limitation, fluorescent, luminescent, or radioactive or they may absorb or reflect visible or ultraviolet light. Examples of detectors suitable for detecting such detectable labels include, without limitation, x-ray film, radioactivity counters, scintillation counters, spectrophotometers, colorimeters, fluorometers, luminometers, photodetectors and densitometers. In certain embodiments the detection agent may comprise a secondary antibody. The expression level is then determined using an unlabelled primary antibody that binds to the target protein and a secondary antibody conjugated to a label, wherein the secondary antibody binds to the primary antibody.

Additional techniques for determining expression level at the level of protein and/or the amount and/or concentration of a marker include, for example, Western blot, immunoprecipitation, immunocytochemistry, mass spectrometry, ELISA and others (see ImmunoAssay: A Practical Guide, edited by Brian Law, published by Taylor & Francis, Ltd., 2005 edition). To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies. Levels of protein may be detected using a lateral flow assay in some embodiments.

Measuring mRNA in a biological sample may be used as a surrogate for detection of the level of the corresponding protein in the wound exudate. Thus, the expression level of any of the relevant markers described herein can also be detected by detecting the appropriate RNA.

Accordingly, in specific embodiments the expression level is determined by microarray, northern blotting, sequencing (including next generation sequencing, such as RNAseq) or nucleic acid amplification. Nucleic acid amplification includes PCR and all variants thereof such as real-time and end point methods and qPCR. Other nucleic acid amplification techniques are well known in the art, and include methods such as NASBA, 3SR and Transcription Mediated Amplification (TMA). Other suitable amplification methods include the ligase chain reaction (LCR), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO 90/06995), invader technology, strand displacement technology, recombinase polymerase amplification (RPA), nicking enzyme amplification reaction (NEAR) and nick displacement amplification (WO 2004/067726). This list is not intended to be exhaustive; any nucleic acid amplification technique may be used provided the appropriate nucleic acid product is specifically amplified. Design of suitable primers and/or probes is within the capability of one skilled in the art. Various primer design tools are freely available to assist in this process such as the NCBI Primer-BLAST tool. Primers and/or probes may be at least 15, 16, 17, 18, 19, 20, 21, 22, 23, 24 or 25 (or more) nucleotides in length. mRNA expression levels may be measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004. RNA expression may be determined by hybridization of RNA to a set of probes. The probes may be arranged in an array. Microarray platforms include those manufactured by companies such as Affymetrix, Illumina and Agilent. RNA expression may also be monitored using next generation sequencing techniques, such as RNA-seq.

Similarly, activity of the one or more markers (e.g. enzymatic activity) may be measured in the wound exudate. Enzymatic activity may be measured for example by detecting processing of a substrate, which may be labelled. For example, the assay may be a fluorogenic substrate assay. Enzyme activity may be detected using a suitable lateral flow assay. Examples of suitable assay formats include the assays set forth in International Patent Applications WO2009/024805, WO2009/063208, WO2007/128980, WO2007/096642, WO2007/096637, WO2013/156794 and WO2013/156795 (the content of each of which is hereby incorporated by reference).

In another aspect of the invention, the methods described herein are repeated at intervals in order to facilitate longitudinal monitoring of the condition of the wound by repeated sampling and analysis of the wound exudate. Thus, for instance, following removal of a product as described herein which has been in contact with the wound for a pre-determined period of time and which has absorbed wound exudate and prior to re-dressing the wound, a new, sterile product as described herein is placed in contact with the wound underneath the new wound dressing and the method repeated in respect of the new product now in contact with the wound. Said intervals may be every 1, 2, 3, 4, 5 or 6 days, weekly or monthly or a combination thereof. As discussed herein, the product may only be sent to the laboratory for further testing if the visual indication of an alteration in the condition of the wound is provided. Nonetheless, by using a new product each time the wound is re-dressed, the initial marker testing is performed on a regularly repeated basis with the consequential benefit that the sample for downstream testing is being obtained on each occasion.

Longitudinal monitoring of the wound exudate in this manner may also be performed even in the absence of a visual indication by the product following contact with the wound for a pre-determined period of time (“pre-determined contact time”). For instance, the product may still be removed from contact with the wound (and replaced with a new, sterile product) and the absorbed wound exudate analysed as described herein if a pre-determined period of time has passed since exudate from the wound has been sampled (“the pre-determined sampling time”). The pre-determined sampling time may be every 1, 2, 3, 4, 5 or 6 days, weekly or monthly or a combination thereof. In a preferred embodiment, the pre-determined sampling time is 4 weeks after the previous sample was taken (in the absence of a visual indication from the product in the intervening period). Thus, for example, the product may be replaced each time the wound is dressed at or around 3 to 4 day intervals. The product may be sent to the laboratory as a matter of routine once a month even if none of the products have shown the visual indication in the intervening period.

The aggregation of data pertaining to the condition of the wound over time via sampling and analysing the wound exudate over time better enables the clinician to understand the progress of the condition of the wound and/or efficacy of treatment(s). For instance, longitudinal monitoring of the wound exudate as described may indicate to the clinician, in a more rapid and/or quantitative fashion than current procedures, that the condition of the wound is deteriorating over time and thus the present treatment is ineffective. Consequently, the clinician can more rapidly select alternative treatments in order to promote healing of the wound. Alternatively, the data may indicate to the clinician that further tests of the wound exudate and/or wound environment are needed. Furthermore, the aggregated data allows the clinician to develop a visiting schedule in relation to further sampling of the wound exudate and re-dressing of the wound by a caregiver, such as a district nurse or family member, depending on the degree and direction of change in the condition of the wound over time.

In a further embodiment, the absence of any visual indication of an alteration in the condition of the wound indicates that existing treatment of the wound should be continued (i.e. should not be altered).

In certain embodiments according to all aspects of the invention, the wound is a chronic wound. A “chronic wound” should be understood to be a wound in which the normal process of wound healing is disrupted at one or more points in the phases of wound healing. For instance, a wound stuck in a particular phase of healing such as inflammation or proliferation. A chronic wound may be characterized by a raised, hyperproliferative, yet non-advancing wound edge and/or a local wound environment, rich in inflammatory products, and proinflammatory cytokines comprising an imbalanced enzymatic milieu consisting of an excess of matrix metalloproteases and a reduction in their inhibitors resulting in the destruction of the extracellular matrix. Common chronic wounds include diabetic ulcers, vascular ulcers and pressure ulcers.

In certain embodiments according to all aspects of the invention, the subject is an animal. In particular embodiments, the animal is a human.

In particular embodiments, a product as described herein is used in a method as described herein.

The invention can also be defined by the following clauses:

1. A product for monitoring the condition of a wound comprising:
(i) a biologically inert matrix which absorbs wound exudate
(ii) one or more reagents on or in the matrix for measuring one or more markers comprised within the wound exudate

wherein a change in the one or more reagents caused by the one or more markers comprised within the wound exudate provides a visual indication of an alteration in the condition of the wound.

2. The product according to clause 1 wherein the one or more reagents comprise a complete test unit integrated on or in the matrix.

3. The product according to any one of clauses 1 or 2 wherein the one or more reagents form a discrete reaction zone on or within the matrix.

4. The product according to any one of clauses 1-3 wherein the alteration is a deterioration.

5. The product according to any one of clauses 1-4 wherein the matrix is able to absorb and retain a volume of wound exudate sufficient for further analysis of the wound exudate.

6. The product according to clause 5 wherein the matrix has the capacity to absorb a volume of at least 0.2 ml wound exudate.

7. The product according to any one of clauses 5 or 6 wherein the matrix has the capacity to absorb a volume up to 10 ml wound exudate.

8. The product according to any one of clauses 5-7 wherein the matrix has the capacity to absorb a volume of 3 ml wound exudate.

9. The product according to any one of clauses 1-8 wherein the matrix is dimensioned to facilitate positioning between a wound dressing and the wound.

10. The product according to clause 9 wherein the dimensions of the matrix are 5 mm×25 mm×25 mm.

11. The product according to clause 9 wherein the dimensions of the matrix are between 2 mm×10 mm×10 mm and 7 mm×40 mm×40 mm.

12. The product according to any of clauses 1-11 wherein the matrix comprises:
(i) a first matrix portion comprising one or more reagents on or in the matrix portion for measuring one or more markers comprised within the wound exudate; and
(ii) a second matrix portion which is able to absorb and retain a volume of wound exudate sufficient for further analysis of the wound exudate.

13. The product according to clause 12 wherein the two matrix portions are laminated together.

14. The product according to any one of clauses 12 or 13 wherein the surface of the first matrix portion that is not in contact with the second matrix portion is coated or surrounded by a transparent protective layer.

15. The product according to clause 12 wherein the two matrix portions are separate, independent components.

16. The product according to any one of clauses 1-15 wherein the matrix does not measurably alter the condition of the exudate or its components once absorbed.

17. The product according to any one of clauses 1-16 wherein the matrix is sufficiently resistant to compression to allow the matrix to maintain a structure suitable to absorb sufficient volumes of wound exudate for further testing.

18. The product according to any one of clauses 1-17 further comprising a reaction vessel extending from the matrix and which is in fluid connection with the matrix.

19. The product according to clause 18 wherein the reaction vessel is connected with the matrix via one or more capillary flow paths.

20. The product according to any one of clauses 18 or 19 wherein the reaction vessel extends sufficiently from the matrix to be positioned outside of a wound dressing applied to the wound such that it is visible to a subject.

21. The product according to any one of clauses 18-20 wherein the reaction vessel absorbs wound exudate indirectly via the matrix.

22. The product according to any one of clauses 18-21 wherein the reaction vessel incorporates one or more reagents for measuring one or more markers comprised within the wound exudate wherein a change in the one or more reagents caused by one or more markers comprised within the wound exudate provides a visual indication of an alteration in the condition of the wound.

23. The product according to any one of clauses 18-22 wherein the one or more reagents incorporated within the reaction vessel is the same as the one or more reagents on or in the matrix.

24. The product according to any one of clauses 18-23 wherein the one or more reagents incorporated within the reaction vessel are different from the one or more reagents on or in the matrix.

25. The product according to clause 24 wherein the one or more reagents incorporated in the reaction vessel and the different one or more markers on or in the matrix measure one or more different markers comprised within the wound exudate respectively.

26. The product according to clause 24 wherein the one or more reagents incorporated in the reaction vessel and the different one or more markers on or in the matrix measure the same one or more markers comprised within the wound exudate.

27. The product according to clause 22 wherein the one or more reagents incorporated within the reaction vessel are in replacement of the matrix reagents such that the reaction vessel provides the only visual indication of an alteration in the condition of the wound and the remainder of the matrix to which the reaction vessel is connected, provides the role of exudate absorbent only.

28. The product according to any one of clauses 18-27 wherein the reaction vessel is closed to the environment.

29. The product according to any one of clauses 18-28 wherein the reaction vessel comprises an absorbent material contained within an impermeable housing.

30. The product according to clause 29 wherein the housing comprises a transparent window.

31. The product according to clause 29 wherein the housing is transparent.

32. The product according to any one of clauses 18-31 wherein the one or more reagents are not biocompatible and are contained within the reaction vessel so as not to be released into the matrix following exposure to wound exudate.

33. The product according to any one of clauses 18-32 wherein the one or more reagents are covalently linked to the reaction vessel and remain so after interaction with one or more markers present in the wound exudate.

34. The product according to any one of clauses 18-33 wherein the reaction vessel further comprises a one-way valve at the point of fluid connection with the matrix such that fluid that has entered the reaction vessel cannot escape back into the matrix.

35. The product according to any one of clauses 1-34 wherein the matrix is composed of a porous material.

36. The product according to any one of clauses 1-35 wherein the matrix comprises, or is composed of, a material selected from any one or more of:
(i) polyurethane;
(ii) polyethylene;
(iii) cellulose fibres; and/or
(iv) porous hydrophilic plastic.

37. The product according to clause 36 wherein, when the product is formed of two matrix portions, the two matrix portions comprise, or are composed of, the same or different materials.

38. The product according to clause 37 wherein the second matrix portion is comprised or composed of polyurethane, optionally polyurethane foam.

39. The product according to any one of clauses 36-38 wherein the polyurethane is a non-isocyanate based polyurethane.

40. The product according to any one of clauses 1-39 wherein the one or more reagents are insoluble in aqueous conditions.

41. The product according to any one of clauses 1-40 wherein the one or more reagents comprise a cross-linked polymer.

42. The product according to any one of clauses 1-41 wherein the one or more reagents are dried into the matrix.

43. The product according to any one of clauses 1-42 wherein the one or more reagents are conjugated to the matrix.

44. The product according to any one of clauses 1-43 wherein the change in the one or more reagents is degradation of the one or more reagents.

45. The product according to clause 44 wherein degradation of the one or more reagents by one or more markers present in the wound exudate reveals a visible symbol in the matrix, otherwise visually concealed by the one or more reagents.

46. The product according to clause 45 wherein the visible symbol is a printed visible symbol.

47. The product according to any one of clauses 1-46 wherein the one or more reagents comprise a:
(i) a protease substrate; and/or
(ii) a myeloperoxidase substrate.

48. The product according to any one of clauses 1-47 wherein the one or more reagents comprise a substrate for matrix metalloprotease collagenase.

49. The product according to any one of clauses 1-48 wherein the one or more reagents are labelled.

50. The product according to any one of clauses 1-49 wherein the one or more reagents are coloured.

51. The product according to any one of clauses 49 or 50 wherein the one or more reagents are dried with:
(i) activated carbon particles; and/or
(ii) coloured micro-particles;

entrained within the dried reagent mass.

52. The product according to clause 51 wherein the micro-particles comprise:
(i) copper phthalocyanine tetrasulfonic acid tetrasodium salt;
(ii) latex microspheres; and/or
(iii) polystyrene microspheres.

53. The product according to any one of clauses 49-52 wherein the one or more reagents are labelled with a fluorescent label that is quenched unless and until the one or more reagents are changed, for instance by cleavage, by one or more markers present in the wound exudate.

54. The product according to any one of clauses 49-53 wherein the one or more reagents are chemically conjugated to one or more dye molecules.

55. The product according to any one of clauses 1-54 wherein the one or more reagents comprise collagen.

56. The product according to clause 55 wherein the collagen is gelatin.

57. The product according to any one of clauses 1-56 wherein the one or more reagents comprise a substrate for a serine protease.

58. The product according to clause 57 wherein the serine protease is neutrophil elastase, optionally human neutrophil elastase.

59. The product according to any one of clauses 1-58 wherein the one or more reagents comprise elastin.

60. The product according to any one of clauses 1-59 wherein the one or more reagents comprise a substrate for a cathepsin protease.

61. The product according to clause 60 wherein the cathepsin protease is cathepsin G.

62. The product according to any one of clauses 1-61 wherein the one or more reagents comprise a substrate for papain-family enzymes, such as staphopain from Staphylococcus aureus.

63. The product according to any one of clauses 1-62 wherein modification of the one or more reagents by exudate from the wound indicates the need for further analysis of the exudate.

64. The product according to any one of clauses 1-63 wherein the absorbed exudate can be retrieved from the matrix for further analysis.

65. The product according to any one of clauses 1-64 wherein the change in the one or more reagents only occurs if the one or more markers are present in the wound exudate at or above a pre-determined threshold level.

66. A wound dressing comprising the product according to any one of clauses 1-65.

67. A kit comprising the product according to any one of clauses 1-66 and a vessel suitable for safe containment and shipping of the product.

68. A kit according to clause 67 wherein, when the matrix comprises two portions and the two portions are provided as two separate components.

69. A kit according to clause 68 wherein the first and second matrix portions can be connected to one another.

70. A kit according to any one of clauses 67-69 wherein the internal surfaces of the vessel are biologically inert.

71. A kit according to any one of clauses 67-69 wherein all surfaces of the vessel are biologically inert.

72. A kit according to any one of clauses 67-71 wherein contact of the matrix with the internal surface of the vessel does not measurably alter the condition of the exudate or its components.

73. A kit according to any one of clauses 67-72 wherein the vessel is sealable.

74. A kit according to clause 73 wherein the sealable vessel comprises a reversible seal.

75. A kit according to any one of clauses 67-74 wherein the vessel is reusable.

76. A method for monitoring the condition of a wound on a subject comprising:
(a) placing a product according to any one of clauses 1 to 66 in contact with the wound under a wound dressing;
(b) leaving the product in contact with the wound for a pre-determined amount of time;
(c) determining the presence or absence of a visual indication of an alteration in the condition of the wound by the product;

wherein the presence of the visual indication signals the need for further analysis of the wound exudate.

77. The method according to clause 76 wherein, when the product comprises a matrix comprising two portions and the two portions are separate components not connected to each other, each portion is independently placed in contact with the wound for a pre-determined amount of time.

78. The method according to clause 76 wherein, when the product comprises a matrix comprising two portions and the two portions are separate components, prior to step (a) the first and second matrix portions are connected to one another.

79. The method according to any one of clauses 76-78 wherein the visual indication is combined with one or more indications selected from:
(i) the smell of the wound
(ii) the total volume of exudate
(iii) the appearance of the wound
(iv) the systemic condition of the subject

in order to determine the need for further analysis of the wound exudate.

80. The method according to any one of clauses 76 or 79 wherein the method further comprises:
(d) removal of the product from contact with the wound;
(e) retrieving the exudate absorbed by the product;
(f) analysing the retrieved exudate in order to determine the condition of the wound.

81. The method according to any one of clauses 76-80 wherein the product used is as defined in any one of clauses 18-66 and the reaction vessel is used to remove the product from the wound.

82. The method according to any one of clauses 76-81 wherein, when the product comprises a matrix comprising a first and second portion, the wound exudate is retrieved from the second portion which is able to absorb and retain a volume of wound exudate sufficient for further analysis of the wound exudate.

83. The method according to any one of clauses 80-82 wherein step (d) further comprises storage of the product in a vessel suitable for safe containment and shipping prior to steps (e) and (f).

84. The method according to clause 83 wherein, when the product comprises a matrix comprising a first and second portion, the second matrix portion is stored in a vessel suitable for safe containment and shipping prior to steps (e) and (f).

85. The method according to any one of clauses 83 or 84 wherein the internal and external surfaces of the vessel are biologically inert.

86. The method according to any one of clauses 83-85 wherein contact of the matrix with the internal surface of the vessel does not measurably alter the condition of the exudate or its components.

87. The method according to any one of clauses 83-86 wherein the vessel containing the product removed from the wound is transported to a laboratory for further analysis of the absorbed exudate.

88. The method according to any one of clauses 76 to 87 wherein analysis of the retrieved exudate comprises measuring the levels and/or activities of one or more of:
(i) P1NP
(ii) acPGP
(iii) A biomarker of neutrophil infiltration

89. The method according to clause 88 wherein the levels of P1NP and acPGP are used to determine a Healing Index Ratio.

90. The method according to any one of clauses 88 or 89 wherein equal levels of P1NP and acPGP indicate healing and/or successful treatment of the wound.

91. The method according to any one of clauses 88 or 89 wherein higher levels of acPGP relative to P1NP indicates inflammation and/or increased risk of infection.

92. The method according to any one of clauses 88 or 89 wherein moderately higher levels of P1NP relative to acPGP indicate a strong healing trajectory.

93. The method according to any one of clauses 88 or 89 wherein much higher levels of P1NP relative to acPGP indicate an increased risk of hypergranulation.

94. The method according to clause 88 wherein the biomarker of neutrophil infiltration is calprotectin.

95. The method according to any one of clauses 76 to 94 wherein analysis of the retrieved exudate further or alternatively comprises measuring the levels of one or more of:
(i) Hydroxylation of lysine and proline residues free in the exudate
(ii) angiogenesis biomarkers
(iii) blood vessel differentiation biomarkers

96. The method according to clause 95 wherein low levels of hydroxylation of lysine and proline residues free in the wound exudate indicate the need for treatment to promote increased blood flow and access of oxygen to the wound.

97. The method according to clause 95 wherein the angiogenesis biomarkers comprise vascular endothelial growth factor.

98. The method according to clause 95 wherein the blood vessel differentiation biomarkers comprise intercellular adhesion molecule.

99. The method according to any one of clauses 97 or 98 wherein low levels of vascular endothelial growth factor and/or intercellular adhesion molecule indicate to treat the wound with externally applied vascular endothelial growth factor supplements.

100. The method according to any one of clauses 76 to 99 wherein analysis of the retrieved exudate further or alternatively comprises determining the nitric oxide status of the wound by measuring the levels and/or activities of one or more of:
(i) inducible nitric oxide synthase;
(ii) carboxymethyllysine; and/or
(iii) arginase.

101. The method according to clause 100 wherein a low nitric oxide status indicates to treat the subject suffering from the wound with nitric oxide therapy or an arginine dietary supplement.

102. The method according to any one of clauses 76 to 101 wherein analysis of the retrieved exudate further comprises measuring the levels of one or more of:
(i) desmosine
(ii) MMP8
(iii) calprotectin
(iv) TIMP1
(v) TIMP2
(vi) A1AT
(vii) interleukin 6

103. The method according to any one of clauses 76 to 102 wherein the method is repeated at intervals in order to facilitate longitudinal monitoring of the condition of the wound.

104. The method according to clause 103 wherein the intervals are daily, weekly or monthly or a combination thereof.

105. The method according to any one of clauses 76 to 104 wherein the absence of any visual indication of an alteration in the condition of the wound indicates that existing treatment of the wound should be continued.

106. The method according to any one of clauses 76 to 105 wherein the wound is a chronic wound.

107. The method according to any one of clauses 76 to 106 wherein the subject is an animal, optionally a human.

108. A product according to any one of clauses 1 to 66 for use in a method according to any one of clauses 76 to 107.

109. A product as defined herein with reference to the accompanying drawings.

110. A wound dressing as defined herein with reference to the accompanying drawings.

111. A method as defined herein with reference to the accompanying drawings.

112. A kit as defined herein with reference to the accompanying drawings.

### DESCRIPTION OF THE FIGURES

FIG. 1A is a schematic illustrating one embodiment of the invention wherein a product as described herein is placed in contact with a wound underneath a wound dressing.

FIG. 1B is a schematic illustrating the generation of a visual indication by a product as described herein following absorption of the wound exudate and modification of the one or more reagents on or in the matrix by one or more markers present in the wound exudate.

FIG. 1C is a schematic illustrating one embodiment of the invention wherein a product as described herein is placed in contact with a wound underneath a wound dressing wherein the product comprises a matrix comprising a first and second portion.

FIG. 1D is a schematic illustrating the generation of a visual indication by a product as described herein comprising a matrix comprising a first and second portion following absorption of the wound exudate and modification of the one or more reagents on or in the first matrix portion by one or more markers present in the wound exudate.

FIG. 2A is a schematic illustrating a further embodiment of the invention wherein a product as described herein further comprises a reaction vessel.

FIG. 2B is a schematic illustrating the generation of a visual indication by a product as described herein comprising a reaction vessel following absorption of the wound exudate and modification of the one or more reagents on or in the matrix and contained in the reaction vessel by one or more markers present in the wound exudate.

FIG. 3 provides a flowchart to illustrate one embodiment of the methods of the invention wherein a product as described herein provides a visual indication of an alteration of a change in the condition of a wound.

FIG. 4 provides a flowchart to illustrate a further embodiment of the methods of the invention wherein the absence of a visual indication by a product as described herein indicates there has been no change in the condition of a wound.

FIG. 5 demonstrates one embodiment of the invention able to detect active papain in a sample using gelatin infiltrated with copper phthalocyanine tetrasulfonic acid tetrasodium salt (CPSS) to form a gelatin-CPSS complex.

FIG. 6 demonstrates one embodiment of the invention able to detect active human neutrophil elastase in a sample using gelatin infiltrated with copper phthalocyanine tetrasulfonic acid tetrasodium salt (CPSS) to form a gelatin-CPSS complex.

FIG. 7 demonstrates one embodiment of the invention able to detect active matrix metallopeptidase 9 in a sample using gelatin infiltrated with copper phthalocyanine tetrasulfonic acid tetrasodium salt (CPSS) to form a gelatin-CPSS complex.

FIGS. 8A-C demonstrate one embodiment of the invention able to detect active papain in a sample using gelatin infiltrated with dyed polystyrene microspheres (PSM) to form a gelatin-PSM complex.

FIGS. 9A-B demonstrate one embodiment of the invention able to detect active matrix metallopeptidase 9 in a sample using gelatin infiltrated with dyed polystyrene microspheres (PSM) to form a gelatin-PSM complex.

FIGS. 10A-B demonstrate one embodiment of the invention able to detect active human neutrophil elastase in a sample using gelatin infiltrated with dyed polystyrene microspheres (PSM) to form a gelatin-PSM complex.

### DETAILED DESCRIPTION OF THE INVENTION

The invention will now be described, without limitation but solely to aid understanding of the invention, by reference to the FIGS.

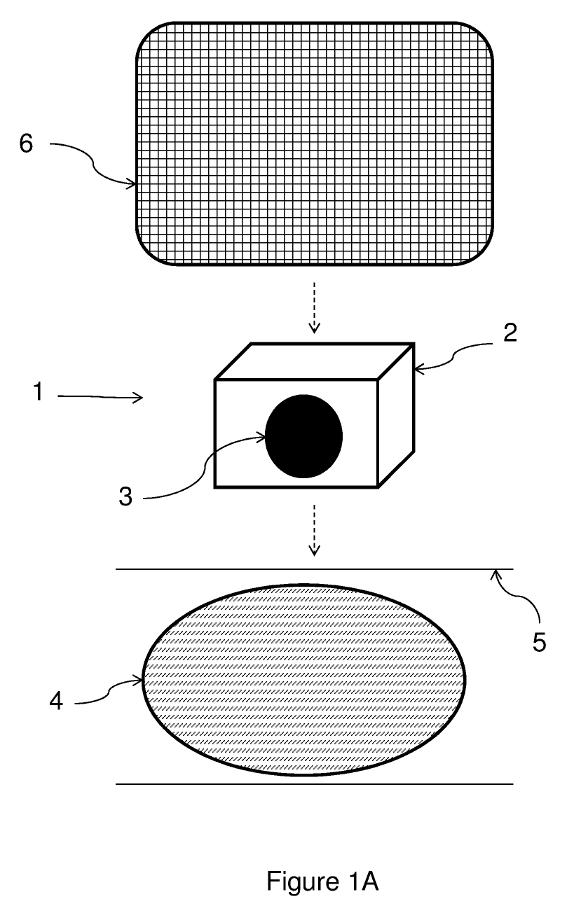

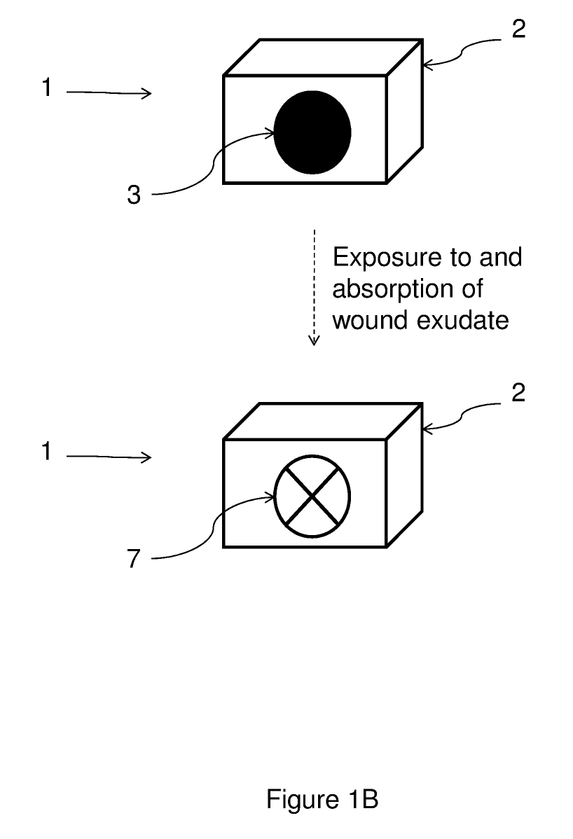

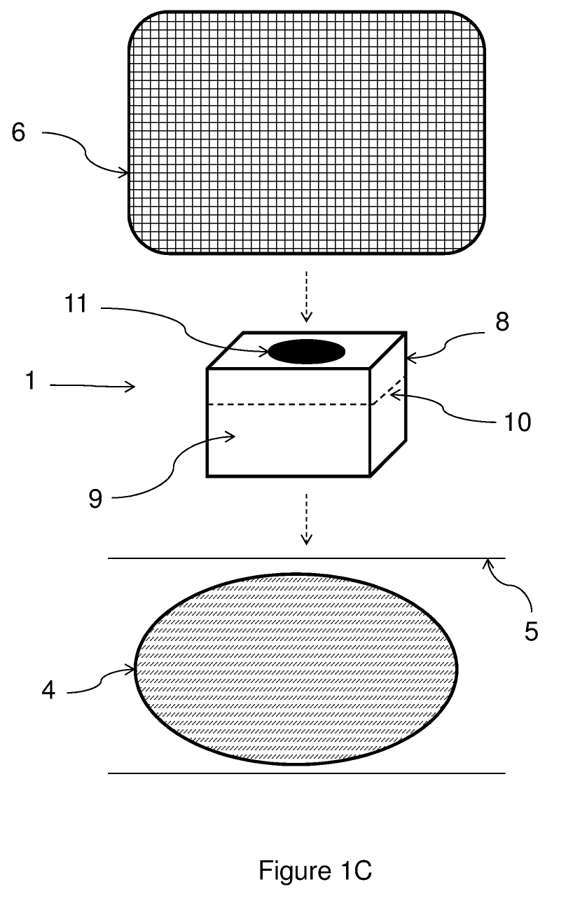

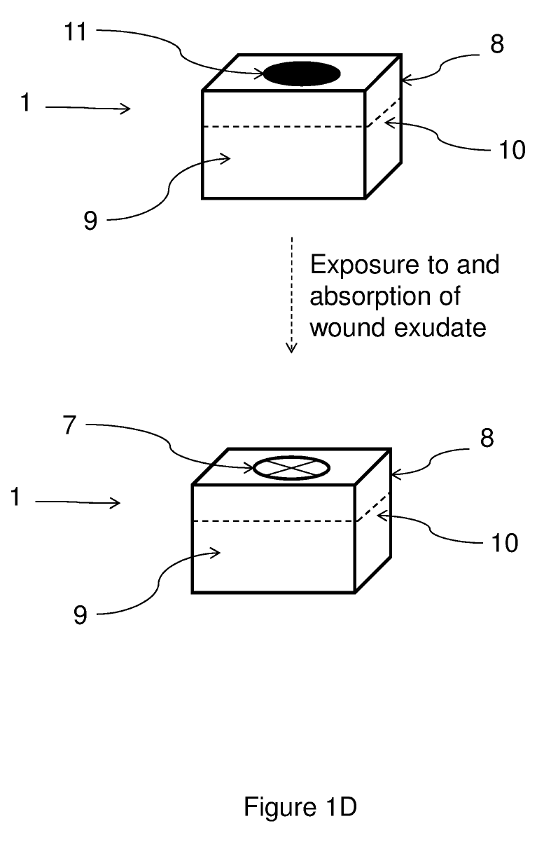

A product (1) as described herein is shown schematically in FIG. 1A. The product (1) comprises a biologically inert matrix (2) which can absorb wound exudate and one or more reagents (3), in this case activated carbon-labelled collagen, on or in the matrix (2). The one or more reagents (3) may be dried into, or conjugated to, the matrix (2) and, in the case of activated carbon-labelled collagen, make the whole or a substantial part of the matrix appear black in colour, forming a test unit or reaction zone. The one or more reagents (3) are used to measure one or more components (markers) comprised within exudate released by a wound. The one or more reagents (3) can be specifically modified by one or more markers of the wound exudate if such markers are present, or present at or above a threshold concentration in the exudate.

When the product (1) is placed in contact with the wound (4), which may be a chronic wound, on a subject (5) underneath a wound dressing (6), the matrix (2) absorbs wound exudate. If the wound exudate comprises one or more markers capable of specifically modifying the one or more reagents this causes a modification in the one or more reagents that consequently provides a visual indication of an alteration in the condition of the wound, such as a deterioration of the wound. In this case, the activated carbon-labelled collagen (3) on or in the matrix (2) is degraded into fragments by collagenase enzymes present in the wound exudate, in particular if the collagenase enzymes are present at or above a threshold level. Once degraded, the fragments of activated carbon-labelled collagen are free to dissociate from the matrix. In this case, as shown in FIG. 1B, dissociation of the activated carbon-labelled collagen fragments from the matrix removes the black colouration that was attributed to the reaction zone of the matrix by the activated carbon label and reveals a visual symbol printed on the product such as a cross (7). The revelation of the visual symbol indicates to the caregiver, such as a district nurse or family member, at the time of re-dressing the wound and replacement of the product (1) with a new, sterile product, that there exists a need for further downstream analysis of the wound exudate such as laboratory testing.

An alternative embodiment is shown in FIGS. 1C and 1D. In this embodiment, the product (1) comprises a first matrix portion (8) and a second matrix portion (9), connected to one another at a connecting surface (10). Both portions are biologically inert and able to absorb wound exudate. The first matrix portion (8) comprises one or more reagents (11), in this case gelatin entrained with coloured polystyrene microspheres (PSM), on or in the first matrix portion (8). The gelatin-PSM complex makes the whole or a substantial part of the first matrix portion appear black in colour, forming a test unit or reaction zone. The one or more reagents (11) are used to measure one or more components (markers) comprised within exudate released by a wound. The one or more reagents (11) can be specifically modified by one or more markers of the wound exudate if such markers are present, or present at or above a threshold concentration in the exudate.

When the product (1) is placed in contact with the wound (4), which may be a chronic wound, on a subject (5) underneath a wound dressing (6), the first and second matrix portions, (8) and (9) respectively, absorb wound exudate. Typically, wound exudate is absorbed by the first matrix portion (8) via fluid connection with the second matrix portion (9) at the connecting surface (10). If the wound exudate comprises one or more markers capable of specifically modifying the one or more reagents this causes a modification in the one or more reagents that consequently provides a visual indication of an alteration in the condition of the wound, such as a deterioration of the wound. In this case, the gelatin of the gelatin-PSM complex (11) entrained within the first matrix portion (8) is degraded into fragments by gelatinase enzymes present in the wound exudate, in particular if the gelatinase enzymes are present at or above a threshold level. Once the gelatin is degraded, the PSM is free to disperse throughout and/or dissociate from the matrix. In this case, as shown in FIG. 1 D, dispersal and/or dissociation of PSM from the matrix dissipates the colouration that was attributed to the reaction zone of the matrix by the gelatin-PSM complex and reveals a visual symbol printed on the product such as a cross (7). The revelation of the visual symbol indicates to the caregiver, such as a district nurse or family member, at the time of re-dressing the wound and replacement of the product (1) with a new, sterile product, that there exists a need for further downstream analysis of the wound exudate such as laboratory testing.

In other embodiments, only the first matrix portion (8) is placed in contact with the wound, thus, the product functions solely as an in-wound protease activity detector. These embodiments are advantageous as they are extremely simple to operate and interpret. In further embodiments, the first matrix portion (8) and second matrix portion (9) are not connected to each other but instead are placed independently in the wound. The first matrix portion (8) provides a visual indication of an alteration in the condition of the wound, such as a deterioration of the wound, as described above. The second matrix portion (9) absorbs wound exudate in an amount sufficient for downstream analysis of the wound exudate.

While the cross (7) provides a positive test result, and is therefore advantageous, it is not essential. Instead dissipation of the colouration can be used as an outcome of the test without revealing a further symbol.

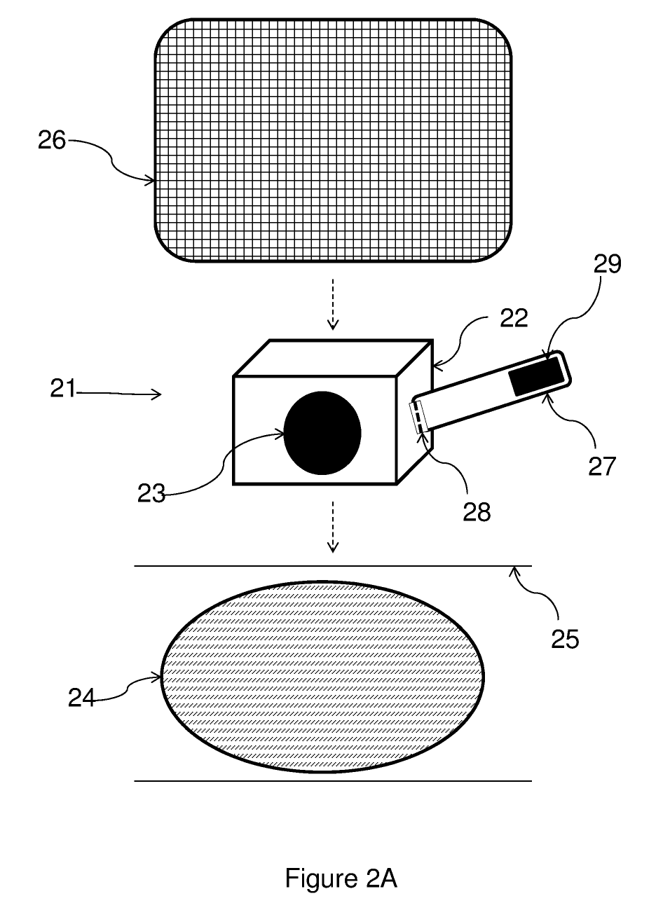

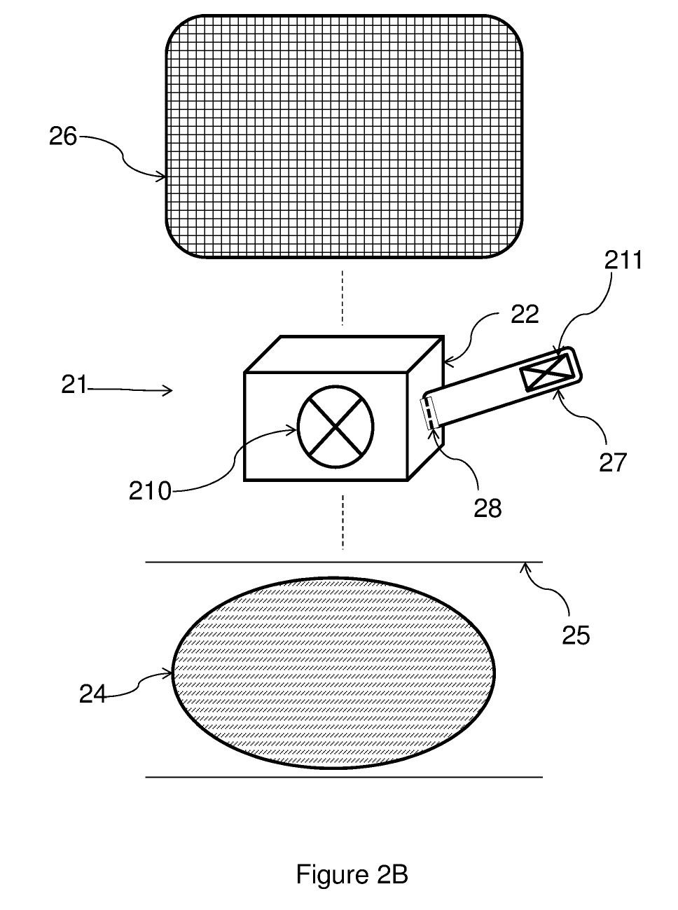

As shown in FIG. 2A, in certain embodiments, the product (21) further comprises a reaction vessel (27) extending from the matrix (22) and which is in fluid connection (28) with the matrix (22), such connection may be via one or more capillary flow paths. The reaction vessel (27) extends sufficiently from the matrix (22) to be positioned at least in part outside of a wound dressing (26) applied to the wound (24) such that it is visible to a subject. The reaction vessel (27) is exposed to and/or may absorb wound exudate by virtue of the fluid connection (28) with the matrix (22) following absorption of wound exudate via the matrix (22). In certain embodiments, the reaction vessel contains one or more reagents (29) able to specifically detect and measure a one or more markers that may be present within the wound exudate. The one or more reagents (29) contained within the reaction vessel (27) may be the same or different to the one or more reagents (23) on or in the matrix (22). In this case, the one or more reagents (23) on or in the matrix (22) and the one or more reagents (29) contained within the reaction vessel (27) is activated carbon-labelled collagen. In other embodiments, the reaction vessel (29) and reagents therein can replace the reagents in the reaction zone of the matrix (23).

In this case, the activated carbon-labelled collagen (23) on or in the matrix (22) is degraded into fragments by collagenase enzymes present in the wound exudate. In addition, exposure of the activated carbon-labelled collagen (29) contained within the reaction vessel (27) to collagenase enzymes present in the wound exudate by virtue of the fluid connection (28) with the matrix (22) degrades the activated carbon-labelled collagen (29) into fragments also. Once degraded, the fragments of activated carbon-labelled collagen are free to dissociate from the reaction vessel and matrix. In this case, as shown in FIG. 2B, dissociation of the activated carbon-labelled collagen fragments from the reaction vessel (27) and matrix (22) removes the black colouration that was attributed to the reaction vessel (27) and matrix (22) by the activated carbon label and reveals a visual symbol, such as a cross, printed on the reaction vessel (211) and matrix (210).

As a consequence of the reaction vessel (27) extending sufficiently from the matrix (22) to be positioned outside of a wound dressing (26), the subject suffering from the wound or the caregiver at the point of care can observe the visual symbol, such as a cross, printed on the reaction vessel (211) without needing to remove the wound dressing. As a result, the subject and/or caregiver is able to receive earlier warning of a change in the condition of the wound, such as deterioration, and can therefore seek clinical intervention more quickly. The inclusion of two detectable reactions provides an internal cross-check.

The presence of the cross in the matrix (210) but not in the reaction vessel (211) may indicate that excess collagenase activity is present but that the volume of exudate absorbed was not sufficient to saturate the reaction vessel (27).

In addition, the reaction vessel (27) provides a handle means by which the product (21) can be removed from the wound (24) without requiring contact with the matrix (22) thereby decreasing the possibility of contamination of the wound exudate absorbed by the matrix (22) via said contact. In some embodiments, the reaction vessel can be cleaved from the matrix (22), for instance at or near to the fluid connection aperture (28), thereby further minimising the possibility of contamination of the wound exudate absorbed by the matrix (22).

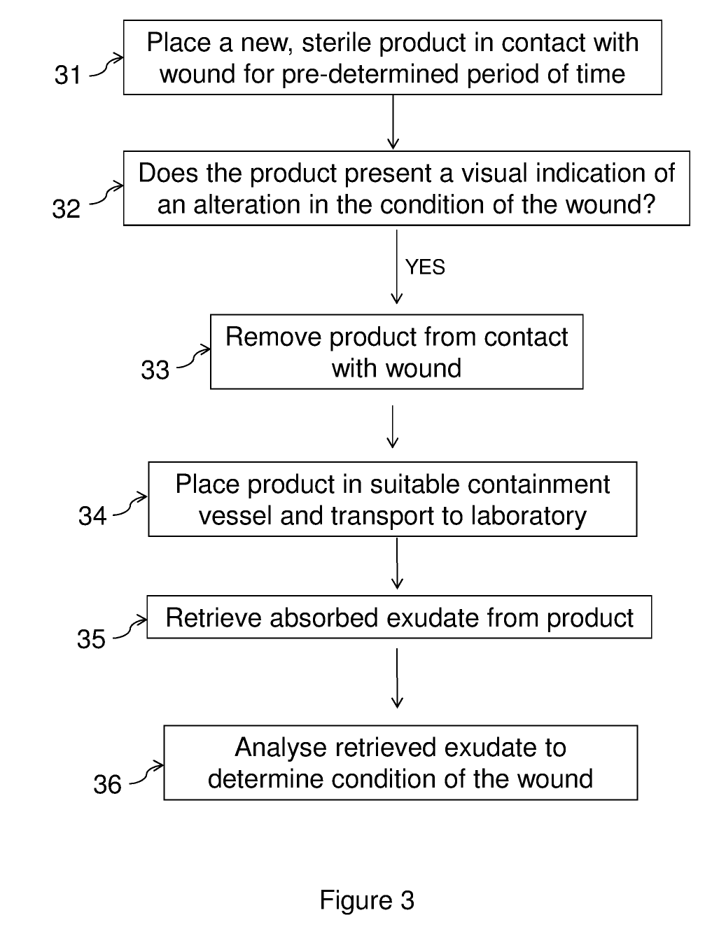

FIG. 3 provides a flowchart to illustrate one embodiment of the methods of the invention. More specifically, a product as described herein, which may, in certain embodiments, be the product as described above in relation to FIG. 1, is placed in contact with a wound on a subject, underneath a wound dressing, for a pre-determined period of time (31). This period of time allows the product to absorb wound exudate.

At the end of this period and at the point of care, the caregiver, such as a district nurse or a family member, removes the wound dressing and assesses the product for the presence of a visual indication by the product as a consequence of a modification in the one or more reagents on or in the product matrix caused by an interaction with one or more markers in the wound exudate (32). For instance, in relation to FIGS. 1A-B, the visual indication may be a symbol such as a cross (7) following degradation of the activated carbon-labelled collagen marker on or in the matrix (2) by collagenase present in the wound exudate that has been absorbed by said matrix, optionally wherein the collagenase enzymes are present at or above a threshold level. Alternatively, in relation to FIGS. 1C-D, the visual indication may be a symbol such as a cross (7) following degradation of the gelatin-PSM complex on or in the matrix (2) by gelatinase present in the wound exudate that has been absorbed by said matrix, optionally wherein the gelatinase enzymes are present at or above a threshold level.

Observance of the visual indication, such as a cross (7), by the caregiver indicates that there has been an alteration in the condition of the wound, which may be deterioration, and that further analysis of the wound exudate is required.

For those embodiments, as described above in relation to FIG. 2, wherein the product (21) further comprises a reaction vessel (27) extending sufficiently from the matrix (22) to be positioned, at least partially, outside of a wound dressing (26) and which is in fluid connection (28) with the matrix (22), the subject suffering from the wound or the caregiver at the point of care can observe the visual symbol, such as a cross, printed on the reaction vessel (211) without needing to remove the wound dressing. For instance, the visual indication, such as a cross (211), provided by the reaction vessel (27) can alert the subject to a change in the condition of the wound prior to any scheduled appointment with a caregiver and/or clinician, thus, enabling the subject to seek earlier clinical intervention and to alert the caregiver and/or clinician that the condition of the wound has altered.

The assessment of the visual indication may be combined with one or more other indications including the smell of the wound, the total volume of wound exudate, the appearance of the wound and/or the systemic condition of the subject in order to determine the need for further analysis of the wound exudate. Thus, the absence of a visual indication by the product in the presence of one or more other indications may still ultimately result in a determination that further analysis of the wound exudate is required.

If further analysis of the wound exudate is required, the product is removed from contact with the wound (33) and placed in a vessel suitable for safe containment and shipping of the product to a laboratory (34). For embodiments where the product comprises a matrix comprising a first and second portion, the second matrix portion and optionally the first matrix portion are placed in a vessel suitable for safe containment and shipping of the product to a laboratory (34). In certain embodiments, such a vessel may comprise a biologically inert internal surface which may not measurably alter the condition of the wound exudate or its components that have been absorbed by the product matrix whilst it was in contact with the wound. In order to minimise contamination of the wound exudate absorbed by the matrix, embodiments of the product comprising a reaction vessel can use said vessel as a handle means to remove the product from contact with the wound. In some embodiments, the reaction vessel can be cleaved from the matrix, for instance at or near to the point of fluid connection with the matrix, so that only the matrix containing the absorbed wound exudate is captured within the containment vessel, thereby further minimising the possibility of contamination of the wound exudate absorbed by the matrix.

The vessel containing the product which itself contains exudate absorbed from the wound is transported to the laboratory where the product may be released from the vessel and the absorbed wound exudate retrieved from the product (35). In some embodiments, the vessel containing the product which itself contains exudate absorbed from the wound is suitable for and is stored in the laboratory under suitable conditions, for instance at −80° C., to allow retrieval and analysis of the wound exudate at a later point in time.

Once the wound exudate is retrieved from the product, the product may be disposed of and the wound exudate analysed to determine the condition of the wound (36).

The process of FIG. 3 may be repeated at intervals in order to facilitate longitudinal monitoring of the condition of the wound. Thus, for instance, following removal of a product which has been in contact with the wound for a pre-determined period of time and which has absorbed wound exudate (33) and prior to re-dressing the wound, a new, sterile product as described herein is placed in contact with the wound underneath the new wound dressing and the process of FIG. 3 repeated. Said intervals may be every 1, 2, 3, 4, 5 or 6 days, weekly or monthly or a combination thereof and may be varied from time to time.

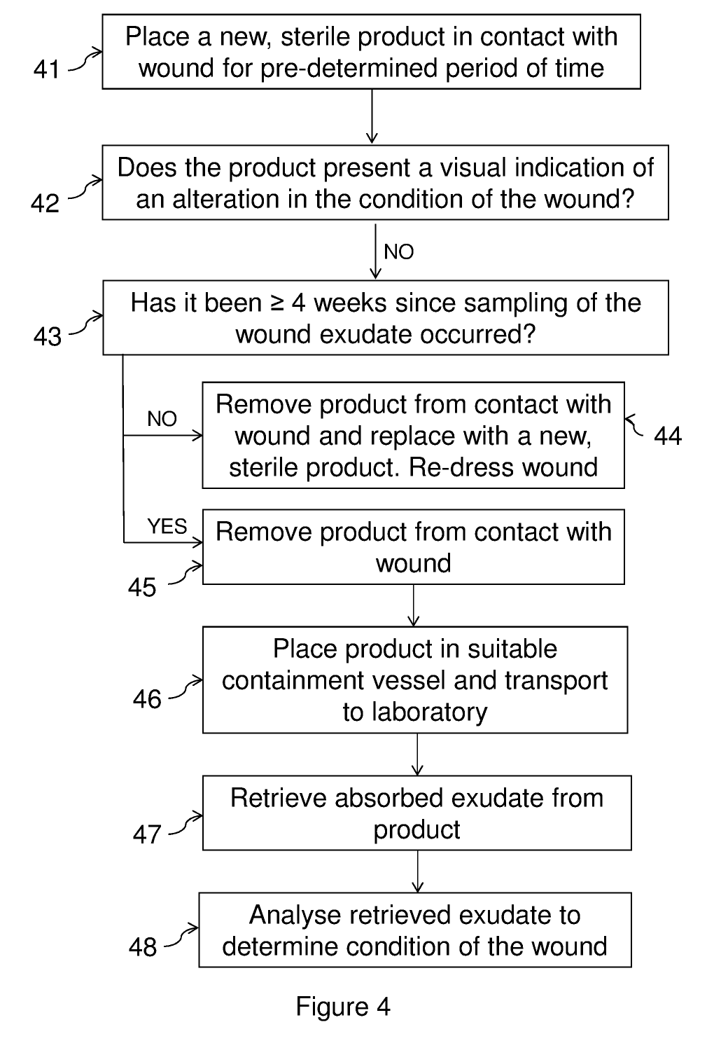

FIG. 4 provides a flowchart to illustrate an alternative outcome in relation to the visual indication and represents particular embodiments of the invention. In this case, a product as described herein is placed in contact with a wound on a subject, underneath a wound dressing, for a pre-determined period of time (41) (“the pre-determined contact time”). This period of time allows the product to absorb wound exudate.

At the end of this period, the product is assessed for the presence of a visual indication by the product as a consequence of a modification in the one or more reagents comprised on or in the product matrix (or contained within the reaction vessel if present) caused by an interaction with one or more markers present in the wound exudate (42).

If no visual indication is presented by the product, the caregiver, which may be a district nurse or a family member, at the point of care may still remove the product from contact with the wound and send the product away for laboratory analysis of the wound exudate (as illustrated (45)-(48) and as described above) if a pre-determined period of time has passed since exudate from the wound has been sampled (“the pre-determined sampling time”), in this case a period of greater than or equal to 4 weeks (43). In alternative embodiments, this period may be every 1, 2, 3, 4, 5 or 6 days, weekly or monthly or a combination thereof. Sampling in this manner facilitates longitudinal monitoring of the condition of the wound. If the pre-determined sampling time has passed and the caregiver removes the product from contact with the wound in order to transport the product to the laboratory for testing of the absorbed wound exudate, the caregiver at the point of care replaces it with a new, sterile product, covers with a fresh wound dressing and monitoring of the wound repeats per FIG. 3 or FIG. 4 as appropriate depending on the presence or absence of a visual indication by the product after the pre-determined contact time. It should be noted that the pre-determined sampling time could be expressed in terms of number of products utilised since the last product was sent for testing. For example, every 5^{th} dressing change the product could be sent for futher testing as described even if no visual indication is presented.

Alternatively, if no visual indication is presented by the product and the pre-determined sampling time has not passed, the caregiver at the point of care removes the product from contact with the wound, replaces it with a new, sterile product, covers with a fresh wound dressing (44) and monitoring of the wound repeats per FIG. 3 or FIG. 4 as appropriate depending on the presence or absence of a visual indication by the product after the pre-determined contact time.

The aggregation of data pertaining to the condition of the wound over time via sampling and analysing the wound exudate over time as described in FIGS. 3 and 4 better enables the clinician to understand the progress of the condition of the wound and efficacy of treatment(s). For instance, longitudinal monitoring of the wound exudate as described may indicate to the clinician, in a more rapid and/or quantitative fashion than current procedures, that the condition of the wound is deteriorating over time and thus the present treatment is ineffective. Consequently, the clinician can more rapidly and in a more informed fashion select alternative treatments in order to promote healing of the wound. Alternatively, the data may indicate to the clinician that further tests of the wound exudate and/or wound environment are needed. Furthermore, the aggregated data allows the clinician to develop a visiting schedule in relation to further sampling of the wound exudate and re-dressing of the wound by a caregiver, such as a district nurse or family member, depending on the degree and direction of change in the condition of the wound over time.

Experimental Section

### EXAMPLE 1

Overview

Gelatin was mixed with Copper Phthalocyanine Tetrasulfonic Acid Tetrasodium salt (CPSS) to form a gelatin-CPSS complex. The gelatin-CPSS sample was dried down onto a support membrane. To initiate the detection of protease activity, the dried gelatin-CPSS sample was wetted with activated protease solution and incubated at room temperature (typically around 21° C.). If zero protease activity was present, the gelatin-CPSS sample remained intact. If protease activity was present, the gelatin was hydrolysed into smaller, mobile fragments, releasing the embedded CPSS which diffused away from the original site of application. This attenuation and dispersal in colour indicated a positive protease reaction.

Experimental Protocol

A 9.1% w/w gelatin (Type A, porcine origin, Sigma G2500) solution in deionised water (DI H₂O) was prepared by adding 1.25 g gelatin powder to 12.5 ml DI H₂O to give a total weight of 13.75 g. The powder was allowed to wet and swell for 5 mins at room temperature (RT) before heating to a minimum of 50° C. The sample was mixed to dissolve the gelatin. 1884 μL of glycerol (Sigma G5516) was added to a final concentration of around 1.2% and thoroughly mixed. The sample was kept at a minimum temperature of 40° C., to ensure the gelatin remained in a liquid state. CPSS (Sigma 274011) powder was added to the liquid gelatin mixture to a final concentration of 2 mg/ml. The sample was mixed to dissolve the CPSS. All CPSS dye was adsorbed by the gelatin and no further processing of the gelatin-CPSS mixture was performed. 2 μL of the mixture was dropped onto a supportive membrane using a calibrated micro-volume pipette (0.5-3 μL). The drop was dried either by air-drying at RT, accelerated using a 37° C. incubator or a drying tunnel at 50° C.

The action of protease enzyme was then evaluated. Examples of proteases used were papain (DMV, around 1000 u/g), human neutrophil elastase (HNE, Lee Biosolutions (code 342-40)) and matrix metalloprotease 9 (MMP9, Alere SD (special commission)). Papain powder was dissolved into DI H₂O at 1 mg/ml, before dilution into activation buffer (1.7 mM EDTA, 10 mM cysteine-HCl, 200 mM sodium chloride, pH 7) to give the required final working concentrations. Stock HNE enzyme was diluted into activation buffer (50 mM Tris, 10 mM calcium chloride dihydrate, 100 mM sodium chloride, 50 μM zinc chloride, 0.025% w/w Brij 35, 0.05% w/w sodium azide, pH 7.4) to give the required final working concentrations. Stock MMP9 enzyme was diluted into activation buffer (50 mM Tris, 10 mM calcium chloride dihydrate, 100 mM sodium chloride, 50 μM zinc chloride, 0.025% w/w Brij 35, 0.05% w/w sodium azide, pH 7.4) to give the required final working concentrations.

Samples of the dry gelatin-CPSS on the support membrane were taken. The membrane was wetted with the specific protease sample until saturation. The samples were left to incubate at RT. At various time points, the integrity of the coloured gelatin-CPSS was examined. A positive result for the presence of protease enzyme was shown by the attenuation of colour due to the digestion of the gelatin and the diffusion of the CPSS molecules away from the site of application. A negative result (i.e. zero active protease) was seen by the original dried gelatin-CPSS indicator remaining in place.

Results

Papain

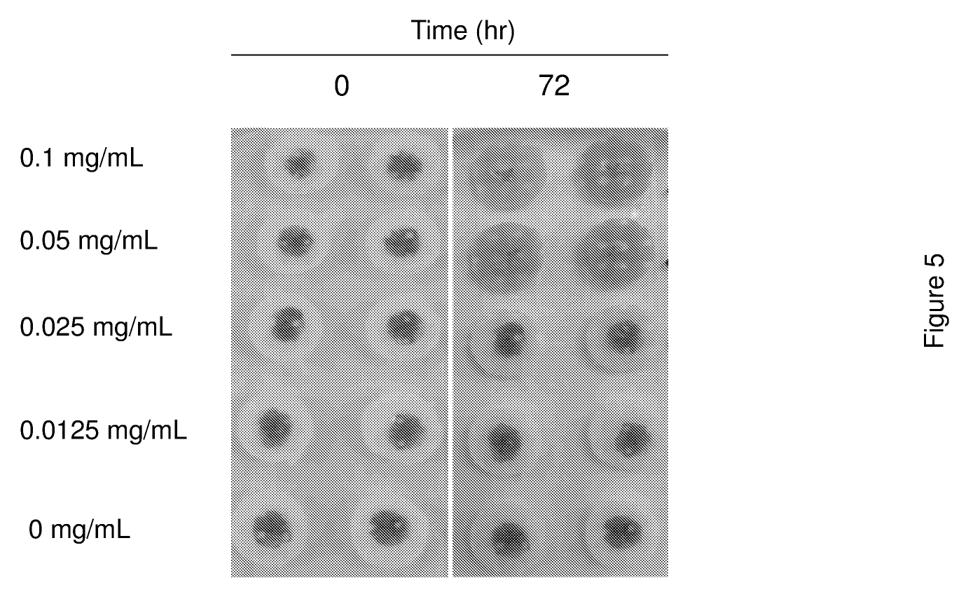

The results in relation to detecting papain activity are shown in FIG. 5.

After 72 hours, papain activity was detected using papain concentrations of 0.1, 0.05 and 0.025 mg/ml. The CPSS molecules diffused throughout the supportive disk resulting in an attenuation of the blue colour and confirming protease activity detection. In the absence of active papain (0 mg/mL), the gelatin-CPSS complex remained intact and no attenuation or dispersal of colour was observed.

HNE

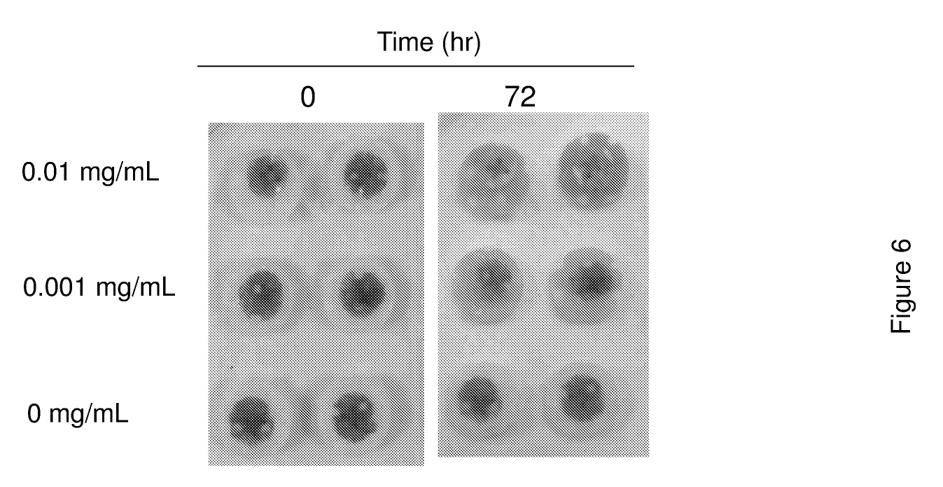

The results in relation to detecting HNE activity are shown in FIG. 6.

After 72 hours, HNE activity was detected using HNE concentrations of 0.01 and 0.001 mg/ml. The CPSS molecules diffused throughout the supportive disk resulting in an attenuation of the blue colour and confirming protease activity detection. In the absence of active HNE (0 mg/mL), the gelatin-CPSS complex remained intact and no attenuation or dispersal of colour was observed.

MMP9

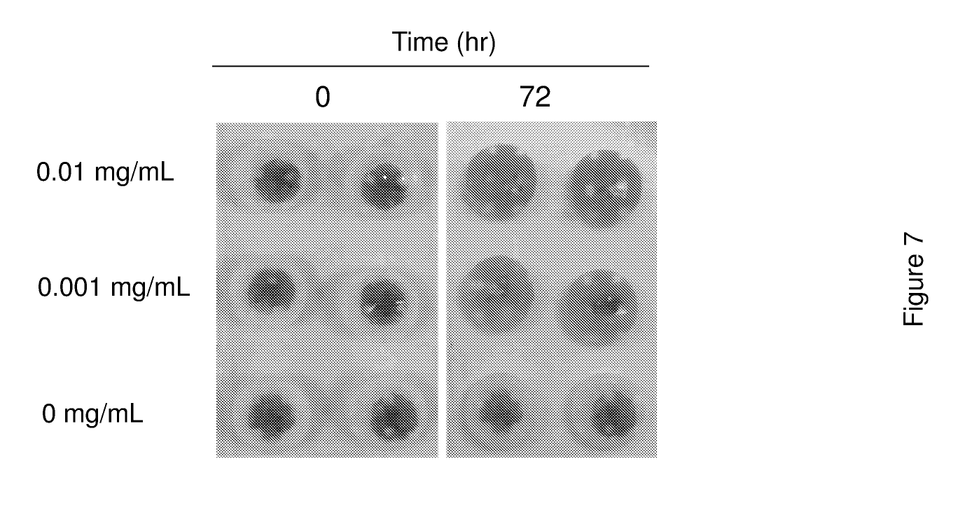

The results in relation to detecting MMP9 activity are shown in FIG. 7.

After 72 hours, MMP9 activity was detected using MMP9 concentrations of 0.01 and 0.001mg/ml. The CPSS molecules diffused throughout the supportive disk resulting in an attenuation of the blue colour and confirming protease activity detection. In the absence of active MMP9 (0 mg/mL), the gelatin-CPSS complex remained intact and no attenuation or dispersal of colour was observed.

Summary

The above examples show the presence of protease activity by the attenuation or loss of the applied coloured spot. This visual change can be interpreted as a negative-read result, due to the loss of colour. The same visual change can also be interpreted as a positive-read result, by the inclusion of an additional permanent coloured mark, either under or above the coloured gelatin-CPSS which acts to hide the mark. When the gelatin-CPSS has been digested and diffused throughout the supportive pad, the masked coloured mark is thus revealed. The revealing of the mark, or “message” can be interpreted as a positive result confirming the presence of protease activity.

### EXAMPLE 2

Overview

Gelatin was mixed with dyed polystyrene microspheres (PSM) to form a gelatin-PSM complex. The gelatin-PSM sample was dried down onto a support membrane. To initiate the detection of protease activity, the dried gelatin-PSM sample was wetted with activated protease solution and incubated at room temperature (typically around 21° C.). If zero protease activity was present, the gelatin-PSM sample remained intact. If protease activity was present, the gelatin was hydrolysed into smaller, mobile fragments, releasing the embedded PSM which diffused away from the original site of application. This attenuation and dispersal in colour indicated a positive protease reaction.

Experimental Protocol

A 9.1% w/w gelatin (Type A, porcine origin, Sigma G2500) solution in deionised water (DI H₂O) was prepared by adding 1.25 g gelatin powder to 12.5 ml DI H₂O to give a total weight of 13.75 g. The powder was allowed to wet and swell for 5 mins at room temperature (RT) before heating to a minimum of 50° C. The sample was mixed to dissolve the gelatin. 188 μL of glycerol (Sigma G5516) was added to a final concentration of around 1.2% and thoroughly mixed. The sample was kept at a minimum temperature of 40° C., to ensure the gelatin remained in a liquid state. Polystyrene microspheres (PSM, 5% solids, 528 nm diameter (blue) or 10% solids, 240 nm (dark blue)) were added to a gelatin solution (3 μL PSM+47 μL gelatin) and mixed to give either 0.3% or 0.6% solids final concentration. 2 μL of the mixture was dropped onto a supportive membrane using a calibrated micro-volume pipette (0.5-3 μL). The drop was dried either by air-drying at RT, or accelerated using a 37° C, incubator or drying tunnel. Protease solutions were prepared as described in Example 5.

Samples of the dry gelatin-PSM on the support membrane were taken. The membrane was wetted with the specific protease sample until saturation. The samples were left to incubate at RT. At various time points, the integrity of the coloured gelatin-PSM was examined. A positive result for the presence of protease enzyme was shown by the attenuation of colour due to the digestion of the gelatin and the diffusion of the PSM molecules away from the site of application. A negative result (i.e. zero active protease) was seen by the original dried gelatin-PSM indicator remaining in place.

Results

Papain

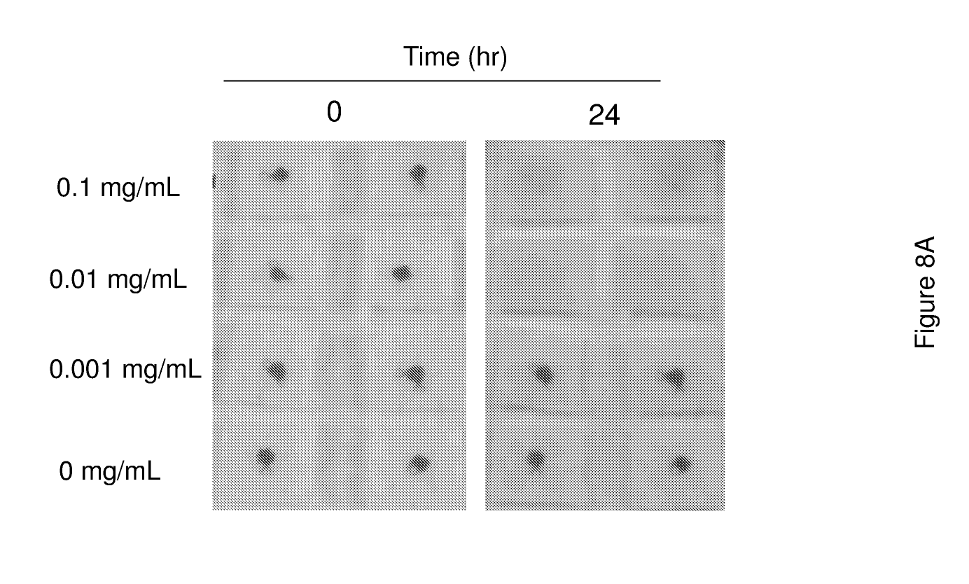

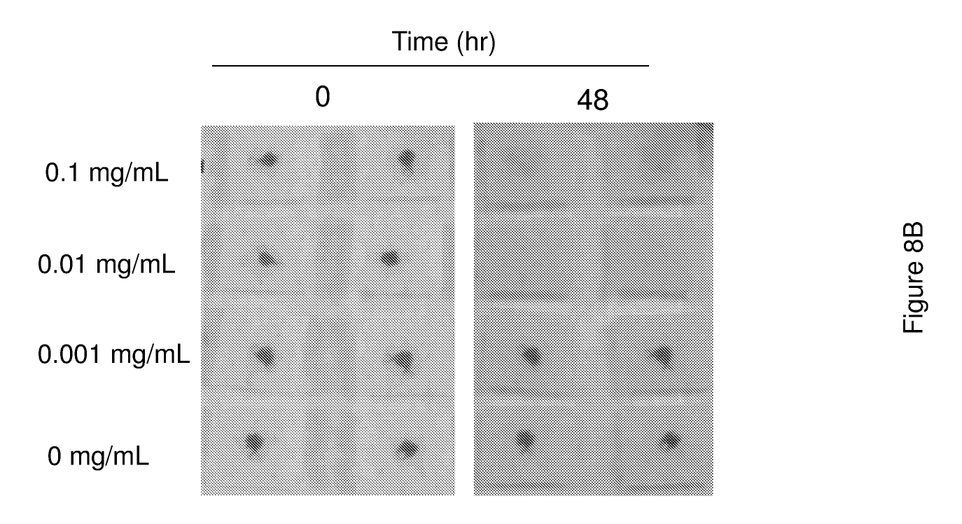

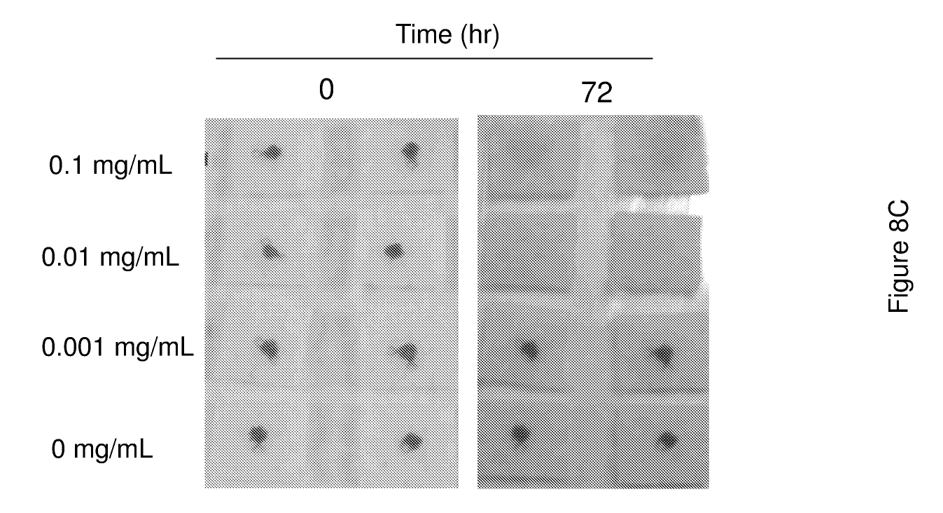

The results in relation to detecting papain activity are shown in FIGS. 8A-C.

After only 24 hours, papain activity was detected using papain concentrations of 0.1 and 0.01 mg/ml. The PSM molecules diffused throughout the supportive disk resulting in an attenuation of the blue colour and confirming protease activity detection. In the absence of active papain (0 mg/mL), the gelatin-PSM complex remained intact and no attenuation or dispersal of colour was observed.

MMP9

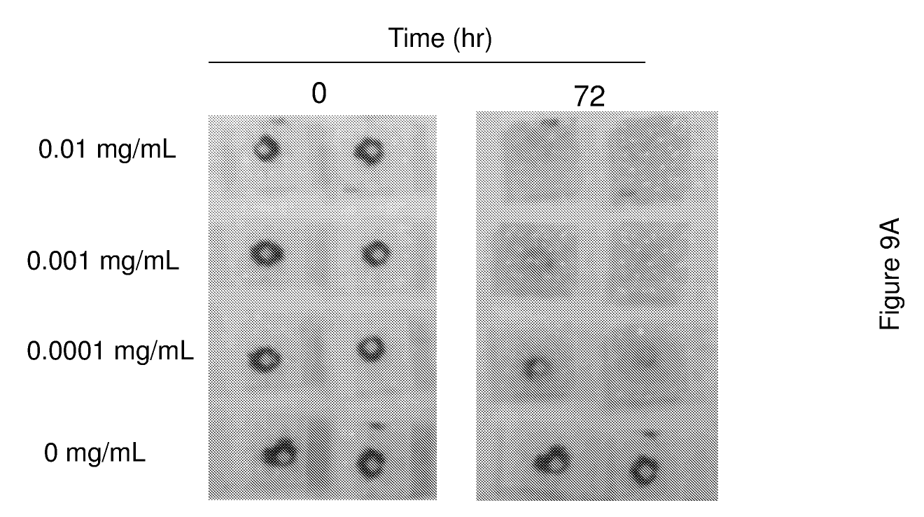

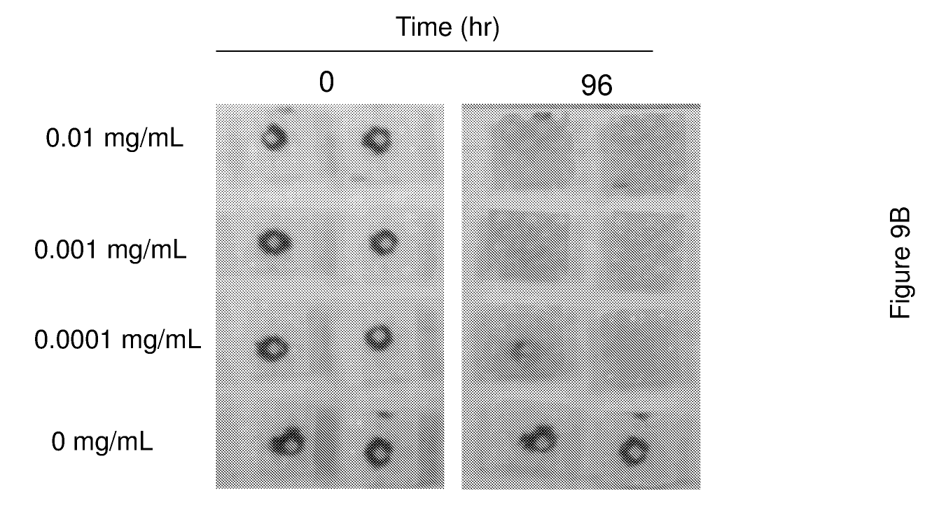

The results in relation to detecting MMP9 activity are shown in FIGS. 9A-B.

After 72 hours, MMP9 activity was detected using MMP9 concentrations of 0.01, 0.001 and 0.0001 mg/ml. The PSM molecules diffused throughout the supportive disk resulting in an attenuation of the blue colour and confirming protease activity detection. In the absence of active MMP9(0 mg/mL), the gelatin-PSM complex remained intact and no attenuation or dispersal of colour was observed.

HNE

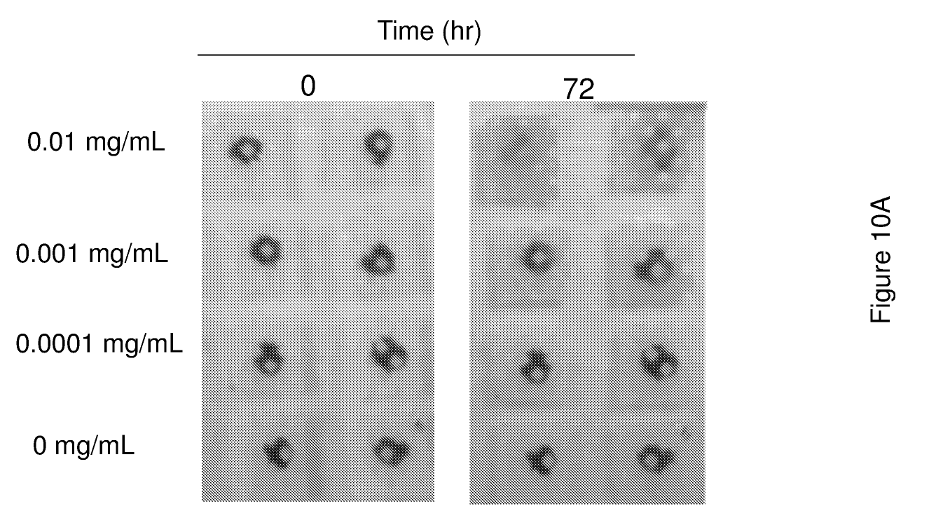

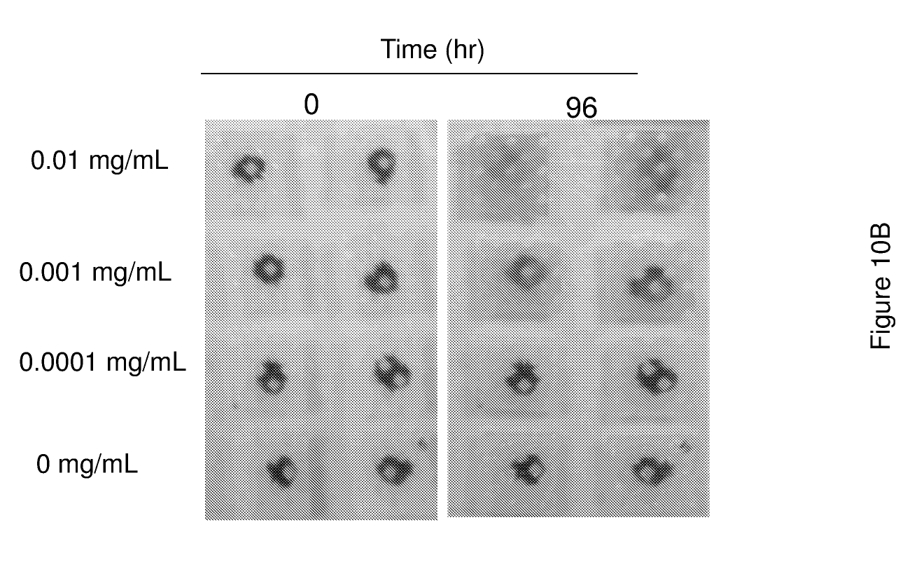

The results in relation to detecting HNE activity are shown in FIGS. 10A-B.

After 72 hours, HNE activity was detected using HNE concentrations of 0.01 and 0.001 mg/ml. The PSM molecules diffused throughout the supportive disk resulting in an attenuation of the blue colour and confirming protease activity detection. In the absence of active HNE (0 mg/mL), the gelatin-PSM complex remained intact and no attenuation or dispersal of colour was observed.

Summary

The above examples show the presence of protease activity by the attenuation or loss of the applied coloured spot. This visual change can be interpreted as a negative-read result, due to the loss of colour. The same visual change can also be interpreted as a positive-read result, by the inclusion of an additional permanent coloured mark, either under or above the coloured gelatin-PSM which acts to hide the mark. When the gelatin-PSM has been digested and diffused throughout the supportive pad, the masked coloured mark is thus revealed. The revealing of the mark, or “message” can be interpreted as a positive result confirming the presence of protease activity.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate. Various publications are cited herein, the disclosures of which are incorporated by reference in their entireties.

1. A product for monitoring the condition of a wound comprising:
(i) a biologically inert matrix which absorbs wound exudate
(ii) one or more reagents on or in the matrix for measuring one or more markers comprised within the wound exudate
wherein a change in the one or more reagents caused by the one or more markers comprised within the wound exudate provides a visual indication of an alteration in the condition of the wound.

2. The product according to claim 1 wherein the one or more reagents comprise a complete test unit integrated on or in the matrix.

3. The product according to claim 1 wherein the one or more reagents form a discrete reaction zone on or within the matrix.

4. The product according to claim 1 wherein the alteration is a deterioration.

5. The product according to claim 1 wherein the matrix is able to absorb and retain a volume of wound exudate sufficient for further analysis of the wound exudate.

6. The product according to claim 5 wherein the matrix has the capacity to absorb a volume of at least 0.2 ml wound exudate.

7. The product according to claim 1 wherein the matrix is dimensioned to facilitate positioning between a wound dressing and the wound.

8. The product according to claim 1 wherein the matrix comprises:
(i) a first matrix portion comprising one or more reagents on or in the matrix portion for measuring one or more markers comprised within the wound exudate; and
(ii) a second matrix portion which is able to absorb and retain a volume of wound exudate sufficient for further analysis of the wound exudate.

9. The product according to claim 8 wherein:
(a) the two matrix portions are laminated together; or
(b) the two matrix portions are separate, independent components.

10. The product according to claim 8 wherein the surface of the first matrix portion that is not in contact with the second matrix portion is coated or surrounded by a transparent protective layer.

11. The product according to claim 1 wherein the matrix does not measurably alter the condition of the exudate or its components once absorbed.

12. The product according to claim 1 wherein the matrix is sufficiently resistant to compression to allow the matrix to maintain a structure suitable to absorb sufficient volumes of wound exudate for further testing.

13. The product according to claim 1 wherein the matrix is composed of a porous material.

14. The product according to claim 1 wherein the matrix comprises, or is composed of, a material selected from any one or more of:
(i) polyurethane;
(ii) polyethylene;
(iii) cellulose fibres; and/or
(iv) porous hydrophilic plastic.

15. The product according to claim 1 wherein, when the product is formed of two matrix portions, the two matrix portions comprise, or are composed of, the same or different materials.

16. The product according to claim 15 wherein the second matrix portion is comprised or composed of polyurethane, optionally polyurethane foam.

17. The product according to claim 14 wherein the polyurethane is a non-isocyanate based polyurethane.

18. The product according to claim 1 wherein:
(a) the one or more reagents are insoluble in aqueous conditions; and/or
(b) the one or more reagents comprise a cross-linked polymer; and/or
(c) the one or more reagents are dried into the matrix; and/or
(d) the one or more reagents are conjugated to the matrix.

19. The product according to claim 1 wherein the change in the one or more reagents is degradation of the one or more reagents.

20. The product according to claim 19 wherein degradation of the one or more reagents by one or more markers present in the wound exudate reveals a visible symbol in the matrix, otherwise visually concealed by the one or more reagents.

21. The product according to claim 1 wherein the one or more reagents comprise:
(i) a protease substrate; and/or
(ii) a myeloperoxidase substrate.

22. The product according to claim 1 wherein:
(a) the one or more reagents comprise a substrate for matrix metalloprotease collagenase; and/or
(b) the one or more reagents are labelled; and/or
(c) the one or more reagents are coloured.

23. The product according to claim 1 wherein the one or more reagents are dried with:
(i) activated carbon particles; and/or
(ii) coloured micro-particles;
entrained within the dried reagent mass.

24. The product according to claim 23 wherein the micro-particles comprise:
(i) copper phthalocyanine tetrasulfonic acid tetrasodium salt;
(ii) latex microspheres; and/or
(iii) polystyrene microspheres.

25. The product according to claim 22 wherein:
(a) the one or more reagents are labelled with a fluorescent label that is quenched unless and until the one or more reagents are changed, for instance by cleavage, by one or more markers present in the wound exudate; and/or
(b) the one or more reagents are chemically conjugated to one or more dye molecules.

26. The product according to claim 1 wherein the one or more reagents comprise collagen, optionally gelatin.

27. The product according to claim 1 wherein:
(a) the one or more reagents comprise a substrate for a serine protease, optionally a neutrophil elastase, in particular a human neutrophil elastase; and/or
(b) the one or more reagents comprise elastin; and/or
(c) the one or more reagents comprise a substrate for a cathepsin protease, optionally cathepsin G; and/or
(d) the one or more reagents comprise a substrate for papain-family enzymes, such as staphopain from Staphylococcus aureus.

28. The product according to claim 1 wherein:
(a) modification of the one or more reagents by exudate from the wound indicates the need for further analysis of the exudate; and/or
(b) the absorbed exudate can be retrieved from the matrix for further analysis; and/or
(c) the change in the one or more reagents only occurs if the one or more markers are present in the wound exudate at or above a pre-determined threshold level.

29. A wound dressing comprising the product according to claim 1.

30. A kit comprising the product according to claim 1 and a vessel suitable for safe containment and shipping of the product.

31. A kit according to claim 30 wherein, when the matrix comprises two portions, the two portions are provided as two separate components, optionally wherein the first and second matrix portions can be connected to one another.

32. A method for monitoring the condition of a wound on a subject comprising:
(a) placing a product according to claim 1 in contact with the wound under a wound dressing;
(b) leaving the product in contact with the wound for a pre-determined amount of time;
(c) determining the presence or absence of a visual indication of an alteration in the condition of the wound by the product;
wherein the presence of the visual indication signals the need for further analysis of the wound exudate.

33. The method according to claim 32 wherein, when the product comprises a matrix comprising two portions and the two portions are separate components not connected to each other, each portion is independently placed in contact with the wound for a pre-determined amount of time.

34. The method according to claim 32 wherein, when the product comprises a matrix comprising two portions and the two portions are separate components, prior to, or as part of, step (a) the first and second matrix portions are connected to one another.

35. The method according to claim 32 wherein the visual indication is combined with one or more indications selected from:
(i) the smell of the wound
(ii) the total volume of exudate
(iii) the appearance of the wound
(iv) the systemic condition of the subject
in order to determine the need for further analysis of the wound exudate.

36. The method according to claim 32 wherein the method further comprises:
(d) removal of the product from contact with the wound;
(e) retrieving the exudate absorbed by the product;
(f) analysing the retrieved exudate in order to determine the condition of the wound.

37. The method according to claim 32 wherein step (d) further comprises storage of the product in a vessel suitable for safe containment and shipping prior to steps (e) and (f).

38. The method according to claim 32 wherein the method is repeated at intervals in order to facilitate longitudinal monitoring of the condition of the wound, optionally wherein the intervals are daily, weekly or monthly or a combination thereof.

39. The method according to claim 32 wherein the absence of any visual indication of an alteration in the condition of the wound indicates that existing treatment of the wound should be continued.

40. The method according to claim 32 wherein the wound is a chronic wound.

41. A product according to claim 1 for use in a method comprising:
(a) placing the product in contact with the wound under a wound dressing;
(b) leaving the product in contact with the wound for a pre-determined amount of time;
(c) determining the presence or absence of a visual indication of an alteration in the condition of the wound by the product;
wherein the presence of the visual indication signals the need for further analysis of the wound exudate.
